(19) United States
(12) Patent Application Publication
(10) Pub. No.: US 2026/0158014 A1
(43) Pub. Date: Jun. 11, 2026

(54) METHODS OF ADMINISTERING VOXELOTOR

(71) Applicant: Global Blood Therapeutics, Inc., South San Francisco, CA (US)

(72) Inventor: Carla B. Washington, South San Francisco, CA (US)

(73) Assignee: Global Blood Therapeutics, Inc., South San Francisco, CA (US)

(21) Appl. No.: 18/707,329

(22) PCT Filed: Nov. 4, 2022
(86) PCT No.: PCT/US2022/079277

§ 371 Date: May 3, 2024

Related U.S. Application Data
(60) Provisional application No. 63/276,498, filed on Nov. 5, 2021.
(60) Provisional application No. 63/286,461, filed on Dec. 6, 2021.
(60) Provisional application No. 63/291,191, filed on Dec. 17, 2021.

(51) Int. Cl. A61K 31/4439; A61K 31/17; A61K 31/4196; A61K 45/06; A61P 7/06

(52) U.S. Cl. CPC A61K 31/4439; A61K 31/17; A61K 31/4196; A61K 45/06; A61P 7/06

(57) ABSTRACT

Provided herein are methods of using voxelotor for the treatment of sickle cell disease in patients, wherein the patient is about 4 years old to less than about 12 years old. Provided herein are methods of using voxelotor for the treatment of sickle cell disease in patients also having severe hepatic impairment. Also provided herein are methods for administering voxelotor and avoiding or lessening adverse drug interactions with a CYP3A4 inducer or inhibitor. Also provided herein are methods for avoiding interactions with voxelotor, a moderate CYP3A4 inhibitor.

### CROSS-REFERENCE TO RELATED APPLICATIONS

This application claims the benefit under 35 U.S.C. § 119(e) of U.S. Provisional Application No. 63/276,498, filed Nov. 5, 2021, U.S. Provisional Application No. 63/286,461, filed Dec. 6, 2021, and U.S. Provisional Application No. 63/291,191, filed Dec. 17, 2021, each of which is hereby incorporated by reference in its entirety.

### FIELD

Provided herein are methods for treating sickle cell disease using voxelotor in patients including patients about 4 years old to less than about 12 years old. Provided herein are methods for treating sickle cell disease using voxelotor in patients that also have severe hepatic impairment. Also provided herein are methods for administering voxelotor and avoiding or lessening adverse drug interactions. Also provided herein are methods for avoiding clinically significant interactions with voxelotor, a moderate CYP3A4 inhibitor.

### BACKGROUND

Voxelotor (also known as Oxbryta® and formerly known as GBT440) is a small molecule allosteric modifier of hemoglobin-oxygen affinity useful for the treatment of sickle cell disease (SCD). Voxelotor can be administered orally. Voxelotor can increase hemoglobin's affinity for oxygen, thereby stabilizing hemoglobin in the oxyhemoglobin state, which can lead to inhibition of polymerization of sickle hemoglobin (HbS).

The major route of elimination of voxelotor is by metabolism. In sickle cell patients with normal hepatic function, 1500 mg of voxelotor significantly increased hemoglobin levels and reduced markers of hemolysis, indicating inhibition of HbS polymerization. In patient populations with severe, moderate, or mild hepatic impairment, drug dosages may or may not need to be adjusted for such patients. Thus, there is a need for developing treatment suitable for SCD patients who also have hepatic impairment.

Further, voxelotor is also a moderate inhibitor of cytochrome P450 (CYP)3A4, the most prevalent CYP enzyme in the liver, in humans. Accordingly, improved methods for administering voxelotor are needed to avoid or lessen potential adverse drug interactions.

### SUMMARY

Heretofore, the therapeutically effective dose of voxelotor for pediatric patients had not been determined. Based on the clinical trial results reported herein, the safe and effective dose of voxelotor is determined by the child's weight. As such, provided herein includes a method of treating sickle cell disease in a patient in need thereof, including patients about 4 years old to less than about 12 years old. Some embodiments provide for a method of treating sickle cell disease in a patient in need thereof, comprising administering to the patient 300 mg to 1000 mg of voxelotor per day, wherein the patient has severe hepatic impairment, the patient is about 4 years old to less than about 12 years old, and the amount of voxelotor is determined by the patient's body weight.

Also provided herein are methods for administering voxelotor and avoiding or lessening adverse drug interactions. Also provided herein are methods for avoiding interactions with voxelotor, a moderate CYP3A4 inhibitor.

### BRIEF DESCRIPTION OF THE DRAWINGS

FIG. 1 shows a forest plot to assess the effect of multiple doses of voxelotor on the pharmacokinetics of caffeine, S-warfarin, omeprazole, midazolam, and their metabolites.

FIG. 2 shows subject-level change from baseline in hemoglobin at week 24 in patients who completed 24 weeks of treatment. Approximately 83% of all randomized patients completed 24 weeks of treatment.

### DETAILED DESCRIPTION

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

It is noted here that as used in this specification and the appended claims, the singular forms “a” “an” and “the” and the like include plural referents unless the context clearly dictates otherwise.

The term “about” or “approximately” means within ±30%, 20%, 15%, 10%, 9%, 8%, 7%, 6%, 5%, 4%, 3%, 2%, 1%, 0.5%, or 0.05% of a given value or range. In some embodiments, “about” means ±5% of a given value or range. In some embodiments, “about” means ±4% of a given value or range. In some embodiments, “about” means ±3% of a given value or range. In some embodiments, “about” means ±2% of a given value or range. In some embodiments, “about” means ±1% of a given value or range. In another embodiment, about means ±0.5% of a given value or range. In some embodiments, “about” means ±0.05% of a given value or range.

As used herein, the term “administration” refers to introducing an agent into a patient. For example, a therapeutic amount can be administered to the patient, which can be determined by the treating physician or the like. In some embodiments, an oral route of administration is preferred. The related terms and phrases “administering” and “administration of,” when used in connection with a compound or tablet (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of an active agent (e.g., voxelotor) administered to a patient in a single day (24-hour period). The desired dose can be administered once daily. In some embodiments, the desired dose may be administered in one, two, three, four or more sub-doses at appropriate intervals throughout the day, where the cumulative amount of the sub-doses equals the amount of the desired dose administered in a single day. The terms “dose” and “dosage” are used interchangeably herein.

As used herein, where the mass of voxelotor is specified, for example, 500 mg, 1000 mg or 1500 mg of voxelotor, that amount corresponds to the mass of voxelotor in its free base form in a single tablet.

The term “hemoglobin” as used herein refers to any hemoglobin protein, including normal hemoglobin (Hb) and sickle hemoglobin (HbS).

The term “sickle cell disease” refers to diseases mediated by sickle hemoglobin (HbS) that results from a single point mutation in the hemoglobin (Hb). Non-limiting examples of sickle cell diseases include sickle cell anemia, sickle-hemoglobin C disease (HbSC), sickle beta-plus-thalassaemia (HbS/β) and sickle beta-zero-thalassaemia (HbS/β0).

As used herein, “therapeutically effective amount” or “therapeutic amount” refers to an amount of a drug or an agent (e.g., voxelotor) that when administered to a patient suffering from a condition, will have the intended therapeutic effect, e.g., alleviation, amelioration, palliation or elimination of one or more manifestations of the condition in the patient. The full therapeutic effect does not necessarily occur by administration of one dose, and can occur only after administration of a series of doses and can be administered in one dose form or multiples thereof. For example, 1000 mg of the drug can be administered in a single 1000 mg strength tablet or two 500 mg strength tablets. As another example, 500 mg of the drug can be administered in a single 500 mg strength tablet. Thus, a therapeutically effective amount may be administered in one or more administrations. For example, and without limitation, a therapeutically effective amount of an agent, in the context of treating disorders related to hemoglobin S, refers to an amount of the agent that alleviates, ameliorates, palliates, or eliminates one or more manifestations of the disorders related to hemoglobin S in the patient.

As used herein, the term “pharmaceutically acceptable” refers to generally safe and non-toxic for in vivo, preferably human, administration.

As used herein, the term “patient” refers to a mammal, such as a human, bovine, rat, mouse, dog, monkey, ape, goat, sheep, cow, or deer. A patient as described herein can be a human. In some embodiments, the patient is an adult. In some embodiments, the patient is a child or juvenile. In some embodiments, the patient is about 12 years old or older. In some embodiments, the patient is about 9 months old to about 11 years old. In some embodiments, the patient is about 4 years old to less than about 12 years old.

As used herein, “treatment,” “treating,” and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition. For purposes of treatment of sickle cell disease, beneficial or desired clinical results include, but are not limited to, multi-lineage hematologic improvement, decrease in the number of required blood transfusions, decrease in infections, decreased bleeding, and the like. For purposes of treatment of interstitial pulmonary fibrosis, beneficial or desired clinical results include, but are not limited to, reduction in hypoxia, reduction in fibrosis, and the like.

As used herein, “% w/w” refers to the weight of a component based on the total weight of a composition comprising the component. For instance, if component 1 is present in an amount of 50% in a 100 mg composition, component 1 is present in an amount of 50 mg. In some embodiments, the composition refers to a tablet as described herein.

### Voxelotor

Voxelotor is a small molecule allosteric modifier of hemoglobin-oxygen affinity in clinical development stage for the treatment of sickle cell disease (SCD). Voxelotor increases hemoglobin's affinity for oxygen, thereby stabilizing hemoglobin in the oxyhemoglobin state, which leads to inhibition of polymerization of sickle hemoglobin. The chemical name for voxelotor is 2-hydroxy-6-((2-(1-isopropyl-1H-pyrazol-5-yl)pyridin-3-yl)methoxy)benzaldehyde. Voxelotor has the structure as shown in the following:

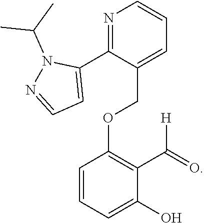 [SMILES: CC(C)n1nccc1-c1ncccc1COc1cccc(O)c1C=O]

The synthesis of voxelotor is described in U.S. Pat. Nos. 9,018,210 and 10,077,249, the disclosures of each of which are incorporated herein by reference in their entireties.

The crystalline solid form of voxelotor includes, for example, crystalline Form I, Form II or Form N as disclosed in PCT Application Publication No. WO 2015/120133, the disclosure of which is incorporated herein by reference in its entirety.

In some embodiments, voxelotor is an ansolvate crystalline form characterized by at least two, three or four X-ray powder diffraction peaks (Cu Kα radiation) selected from 13.37°, 14.37°, 19.950 and 23.92° 2θ (each ±0.2 °2θ). In some embodiments, voxelotor is an ansolvate crystalline form characterized by X-ray powder diffraction peaks (Cu Kα radiation) at 13.37°, 14.37°, 19.950 and 23.92° 2θ (each ±0.2 °2θ). This form can hereinafter also be referred to as Form II.

In some embodiments, voxelotor is an ansolvate crystalline form characterized by at least two, three or four X-ray powder diffraction peaks (Cu Kα radiation) selected from 11.65°, 11.85°, 12.08°, 16.70°, 19.65° and 23.48 °2θ (each ±0.2 °2θ). In some embodiments, voxelotor is an ansolvate crystalline form characterized by X-ray powder diffraction peaks (Cu Kα radiation) at 11.65°, 11.85°, 12.08°, 16.70°, 19.65° and 23.48 °2θ (each ±0.2 °2θ). This form can hereinafter also be referred to as Form N or Material N.

In some embodiments, voxelotor is an ansolvate crystalline form characterized by at least two, three or four X-ray powder diffraction peaks (Cu Kα radiation) selected from 12.82°, 15.74°, 16.03°, 16.63°, 17.60°, 25.14°, 25.82, and 26.44° °2θ (each ±0.2 °2θ). In some embodiments, voxelotor is an ansolvate crystalline form characterized by X-ray powder diffraction peaks (Cu Kα radiation) at 12.82°, 15.74°, 16.03°, 16.63°, 17.60°, 25.14°, 25.82, and 26.44 °2° (each ±0.2 °2θ). This form can hereinafter also be referred to as Form I.

### Methods of Treatment

Provided herein are methods of treating sickle cell disease in a patient in need thereof, comprising administering to the patient 600 mg to 1500 mg of voxelotor per day, wherein the patient is about 4 years old to less than about 12 years old, and the amount of voxelotor is determined by the patient's body weight.

In some embodiments, the patient's body weight is about 40 kg or greater (e.g., greater than about 40 kg), and the patient is administered 1500 mg of voxelotor per day. In some embodiments, the patient's body weight is about 20 kg to less than about 40 kg, and the patient is administered 900 mg of voxelotor per day. In some embodiments, the patient's body weight is about 10 kg to less than about 20 kg, and the patient is administered 600 mg of voxelotor per day. In some embodiments, the administration is once daily.

In some embodiments, the patient may suffer from pyrexia, vomiting, rash, abdominal pain, diarrhea, and headache. In some embodiments, the patient may suffer from vomiting.

In some embodiments, methods described herein further comprise administering to the patient hydroxyurea.

### Hepatic Impairment

As used herein, a subject with “hepatic impairment” is a subject with a reduced hepatic function, for example a subject diagnosed with a clinical decrease in liver function. The reduced liver function can be caused by a liver disease suffered by the subject, for example hepatic encephalopathy, hepatitis, or cirrhosis. Sometimes, hepatic impairment can lead to liver failure. A number of methods have been developed to quantify hepatic functions and to determine the extent of hepatic impairment in patients, including a model end stage liver disease (MELD) score and a Child-Pugh score.

The Child-Pugh score (also known as the Child-Turcotte-Pugh score) is the most commonly used method to assess the prognosis of chronic liver disease, for example cirrhosis. It is an aggregate score of five clinical measures of liver disease: total bilirubin, serum albumin, prothrombin time prolongation or international normalized ratio (INR), ascites, and hepatic encephalopathy. Each marker is assigned a value from 1-3 points, with 3 indicating the most severe derangement. The total value of points is used to provide a score categorized as A (5-6 points), B (7-9 points), or C (10-15 points), which can be correlated with one and two year survival rates. Category A (i.e. Child-Pugh A score of 5-6 points) is considered as mild hepatic impairment, category B is considered to be moderate hepatic impairment (i.e. Child-Pugh B score of 7-9 points), and category C is considered to be severe hepatic impairment (i.e. Child-Pugh B score of 10-15 points). Methods for determination and analysis of Child-Pugh scores are well known in the art. Another commonly used scoring system to assess hepatic impairment, the Model for End-stage Liver Disease (MELD) was initially created to predict survival among patients undergoing transjugular intrahepatic portosystemic shunt (TIPS) placement. The MELD contains three objective variables: international normalized ratio (INR), creatinine, and total bilirubin. More recent but not uniformly adopted modification includes addition of sodium values.

As disclosed herein, any methods recognized in the art suitable to determine the severity of hepatic impairment of a subject can be used. In some embodiments, the hepatic impairment of the patient is determined by the Child-Pugh score. The patient, for example, can have a Child-Pugh score of, or about, 7, 8, 9, 10, 11, 12, 13, 14, 15, or a range between any two of these values, points. In some embodiments, the patient has a Child-Pugh score of, or about, 10, 11, 12, 13, 14, 15, or a range between any two of these values, points. In some embodiments, the patient has a Child-Pugh score of 10-15 points (i.e. category C).

Hepatic impairments in the patients can be caused by various conditions and reasons including, but not limited to liver diseases, cancers, autoimmune diseases, toxins, metabolic diseases, shock (e.g., sepsis, hepatic shock), vascular diseases (e.g., Budd-Chiari syndrome), hypoxemia, medication uses or overuses, and any combinations thereof. For example, the hepatic impairment of the patient can be caused by a liver disease, for example a chronic liver disease or an acute liver disease. The liver diseases can be caused by, for example, infections (e.g., parasite and/or viral infections), immune system abnormality, genetic abnormality, cancer and other growths, chronic alcohol abuse, and/or accumulation of fat in the liver. Examples of liver diseases include, but are not limited to, hepatitis (e.g., hepatitis A, hepatitis B, hepatitis C, hepatitis D, and hepatitis E), autoimmune hepatitis, alcoholic hepatitis, primary biliary cirrhosis, primary sclerosing cholangitis, hemochromatosis, hyperoxaluria and oxalosis, Wilson's disease, alpha-1 antitrypsin deficiency, liver cancer, bile duct cancer, liver adenoma, fatty liver disease (including alcoholic fatty liver disease and nonalcoholic fatty liver disease), cirrhosis, hypo-perfusion, or any combination thereof. In some embodiments, the hepatic impairment of the patient is caused by a physical injury to the liver. In some embodiments, the hepatic impairment of the patient is caused by liver inflammation. In some embodiments, the hepatic impairment of the patient is caused by the use or excess use of one or more drugs, for example acetaminophen, narcotic-acetaminophen combination medications, statin type of drugs for controlling elevated blood levels of cholesterol, niacin, antibiotics (e.g., nitrofurantoin, amoxicillin and clavulanic acid, tetracycline, and isoniazid), drugs for treating autoimmune disorders and/or cancers (e.g., methotrexate), drugs for treating alcoholics (e.g., disulfiram), or any combination thereof. In some embodiments, the hepatic impairment of the patient is caused by a use or an excess use of one or more herbal supplements, including but not limited to, kava, ephedra, skullcap and pennyroyal. In some embodiments, the hepatic impairment of the patient is caused by an excess use of vitamins (e.g., vitamin A). In some embodiments, the hepatic impairment of the patient is caused by food (e.g., mushrooms).

The chronic liver disease can be, for example, liver fibrosis, cirrhosis, end-stage liver disease (ESLD), hepatocellular carcinoma, hepatic steatosis (i.e., fat in the liver), nonalcoholic fatty liver disease (NAFLD), nonalcoholic steatohepatitis (NASH), portal hypertension, or any combination thereof. In some embodiments, the hepatic impairment is caused by chronic liver injury. In some embodiments, the hepatic impairment is caused by hepatic inflammation. Some patients can have ascites, bleeding diathesis, variceal bleeding, hepatic inflammation, renal dysfunction, hepatic encephalopathy, increased susceptibility to infection, and multi-organ dysfunction. The cirrhosis can be compensated or decompensated. In some embodiments, the patient having severe hepatic impairment suffers from a chronic hepatic disease. In some embodiments, the patient having severe hepatic impairment suffers from a chronic hepatic injury.

In some embodiments, the method further comprises identifying a patient having sickle cell disease that also has severe hepatic impairment. For example, clinical data or test results from a patient can be used to determine if a patient has sickle cell disease and severe hepatic impairment. In some embodiments, the identification can be made by a medical professional by using information obtained from the patient, information obtained from the patient's medical records, or information collected from test results. A medical professional having this information available to them and being able to identify subjects having sickle cell disease and severe hepatic impairment can practice the methods disclosed herein.

Methods of Treatment for Patients with Hepatic Impairment

Whether dose adjustment is needed in patients with hepatic impairment can be difficult to predict. Further, the amount of dose adjustment needed is also difficult to predict, including for mild, moderate, or severe hepatic impairment patients.

It is contemplated that the dose of voxelotor for patients, including patients about 4 years old to less than about 12 years old, with normal liver function may need to be adjusted for patients with hepatic impairment. Disclosed herein include methods for using voxelotor for the treatment of sickle cell disease in patients also having hepatic impairment, for example severe hepatic impairment.

In some embodiments, the patient suffers from sickle cell disease and liver disease. In some embodiments, the patient has sickle cell disease and severe hepatic impairment. In some embodiments, the patient has sickle cell disease and moderate hepatic impairment. In some embodiments, the patient has sickle cell disease and mild hepatic impairment.

In some embodiments, the patient can also be a patient suffering from sickle cell disease and a condition that can cause a severe hepatic impairment as described herein. For example, the condition can be cancer, liver injury, autoimmune disease, drug overuse, or any combination thereof. The patient's age can vary. For example, the patient can be an adult, a child or juvenile. In some embodiments, the patient is at least 18 years old. In some embodiments, the patient is at least 12 years old. In some embodiments, the patient is about 6 months old to about 11 years old. In some embodiments, the patient is about 4 years old to less than about 12 years old.

In some embodiments, the patient suffers from a condition selected from the group consisting of liver fibrosis, cirrhosis, hepatocellular carcinoma, hepatic inflammation, hepatic steatosis, nonalcoholic fatty liver disease (NAFLD), nonalcoholic steatohepatitis (NASH), portal hypertension, hepatic encephalopathy, hepatitis, and any combination thereof. In some embodiments, the patient has a Child-Pugh score of 10-15 points (Child-Pugh C). In some embodiments, the patient suffers from a chronic liver disease.

In some embodiments, the methods described herein comprise administering to the patient a therapeutically effective amount of voxelotor. The administration can be oral administration, for example in the form of capsules or tablets. The administration can be, for example, once daily, twice or three times a day, or once every two days. In some embodiments, the administration is once daily. In some embodiments, the methods comprise administering to the patient one or two tablets described herein comprising a therapeutically effective amount of voxelotor per day.

In some embodiments, the dose of voxelotor is reduced to avoid a treatment-emergent adverse event. In some embodiments, the treatment-emergent adverse event is diarrhea. In some embodiments, the treatment-emergent adverse event is a headache. In some embodiments, the treatment-emergent adverse event is nausea, arthralgia, an upper respiratory tract infection, abdominal pain, fatigue, rash, pyrexia, pain in extremity, back pain, vomiting, pain, noncardiac chest pain, or upper abdominal pain. In some embodiments, the treatment-emergent adverse event is abdominal pain, diarrhea, nausea, fatigue, or pain. In some embodiments, the treatment-emergent adverse event is diarrhea, abdominal pain, nausea, fatigue, rash, or drug hypersensitivity.

In some embodiments, the method comprises administering to the patient one to three tablets described herein, wherein each of the one to three tablets comprises about 500 mg of voxelotor and wherein the administration is 1 to 3 times daily. In some embodiments, the method comprises administering to the patient two tablets described herein once daily, wherein each of the two tablets comprises about 500 mg of voxelotor.

In some embodiments, the methods comprise administering to the patient 500 mg to 1000 mg of voxelotor per day. In some embodiments, the methods comprise administering to the patient 500 mg of voxelotor per day. In some embodiments, the methods comprise administering to the patient 1000 mg of voxelotor per day. In some embodiments, the methods comprise administering to the patient one tablet of voxelotor per day, wherein the tablet comprises 500 mg voxelotor. In some embodiments, the methods comprise administering to the patient two tablets of voxelotor per day, wherein each of the two tablets comprises 500 mg voxelotor. In some embodiments, the methods comprise administering to the patient two tablets of voxelotor at once per day, wherein each of the two tablets comprises 500 mg voxelotor. In some embodiments, the methods comprise administering to the patient in a single oral dose of two tablets of voxelotor daily, wherein each of the two tablets comprises 500 mg voxelotor.

Some embodiments provide for methods of treating sickle cell disease in a patient in need thereof, comprising administering to the patient 1000 mg of voxelotor once daily, wherein the patient has severe hepatic impairment.

Some embodiments provide for methods of treating sickle cell disease in a patient in need thereof, comprising administering to the patient 1500 mg of voxelotor once daily, wherein the patient has mild or moderate hepatic impairment.

Some embodiments provide for methods of treating sickle cell disease in a patient in need thereof, comprising administering to the patient 300 mg to 1000 mg of voxelotor per day, wherein the patient has severe hepatic impairment, the patient is about 4 years old to less than about 12 years old, and the amount of voxelotor is determined by the patient's body weight.

In some embodiments, the patient's body weight is about 40 kg or greater (e.g., greater than about 40 kg), and the patient is administered 900 mg of voxelotor per day.

In some embodiments, the patient's body weight is about 40 kg or greater (e.g., greater than about 40 kg), and the patient is administered 1000 mg of voxelotor per day.

In some embodiments, the patient's body weight is about 20 kg to less than about 40 kg, and the patient is administered 600 mg of voxelotor per day.

In some embodiments, the patient's body weight is about 10 kg to less than about 20 kg, and the patient is administered 300 mg of voxelotor per day.

Some embodiments provide for methods of treating sickle cell disease in a patient in need thereof, comprising administering to the patient 1000 mg of voxelotor per day, wherein the patient has severe hepatic impairment, the patient is about 4 years old to less than about 12 years old, and the patient's body weight is about 40 kg or greater (e.g., greater than about 40 kg).

Some embodiments provide for methods of treating sickle cell disease in a patient in need thereof, comprising administering to the patient 900 mg of voxelotor per day, wherein the patient has severe hepatic impairment, the patient is about 4 years old to less than about 12 years old, and the patient's body weight is about 40 kg or greater (e.g., greater than about 40 kg).

Some embodiments provide for methods of treating sickle cell disease in a patient in need thereof, comprising administering to the patient 600 mg of voxelotor per day, wherein the patient has severe hepatic impairment, the patient is about 4 years old to less than about 12 years old, and the patient's body weight is about 20 kg to less than about 40 kg.

Some embodiments provide for methods of treating sickle cell disease in a patient in need thereof, comprising administering to the patient 300 mg of voxelotor per day, wherein the patient has severe hepatic impairment, the patient is about 4 years old to less than about 12 years old, and the patient's body weight is about 10 kg to less than about 20 kg.

Some embodiments provide for methods of treating sickle cell disease in a patient in need thereof, comprising administering to the patient 600 mg to 1500 mg of voxelotor once daily, wherein the patient has mild or moderate hepatic impairment, the patient is about 4 years old to less than about 12 years old, and the amount of voxelotor is determined by the patient's body weight.

In some embodiments, the patient's body weight is about 40 kg or greater (e.g., greater than about 40 kg), and the patient is administered 1500 mg of voxelotor per day. In some embodiments, the patient's body weight is about 20 kg to less than about 40 kg, and the patient is administered 900 mg of voxelotor per day. In some embodiments, the patient's body weight is about 10 kg to less than about 20 kg, and the patient is administered 600 mg of voxelotor per day.

Some embodiments provide for methods of treating sickle cell disease in a patient in need thereof, comprising administering to the patient 1500 mg of voxelotor once daily, wherein the patient has mild or moderate hepatic impairment, the patient is about 4 years old to less than about 12 years old, and the patient's body weight is about 40 kg or greater (e.g., greater than about 40 kg).

Some embodiments provide for methods of treating sickle cell disease in a patient in need thereof, comprising administering to the patient 900 mg of voxelotor once daily, wherein the patient has mild or moderate hepatic impairment, the patient is about 4 years old to less than about 12 years old, and the patient's body weight is about 20 kg to less than about 40 kg.

Some embodiments provide for methods of treating sickle cell disease in a patient in need thereof, comprising administering to the patient 600 mg of voxelotor once daily, wherein the patient has mild or moderate hepatic impairment, the patient is about 4 years old to less than about 12 years old, and the patient's body weight is about 10 kg to less than about 20 kg.

Duration of the treatment can vary. For example, voxelotor can be administered to the patient for at least about, or for about, 20, 21, 22, 23, 24, 25, 26, 27, 28, 29, 30, 31, 32, 33, 34, 35, 36, or a range between any two of these values, days.

In some embodiments, voxelotor (e.g., the tablet(s) of voxelotor) is administrated to the patient with food. In some embodiments, voxelotor (e.g., the tablet of voxelotor) is administrated to the patient without food. In some embodiments, voxelotor is administered to the patient in a single oral dose of one tablet daily, two tablets daily, or three tablets daily.

### Methods of Treatment to Avoid Drug-Drug Interactions (“DDI”)

The cytochrome P450 (CYP) is a superfamily of enzymes responsible for the metabolic transformation of drugs, with CYP3A4 being the most prevalent CYP enzyme in the liver. CYP3A4 activity can be induced (or accelerated) or inhibited (decreased), which can impact drug concentrations. The inhibition of CYP3A4 can result in the accumulation of parent drug concentrations that can put the patient at increased risk for side effects and possible toxicity.

It has been found that voxelotor is a moderate inhibitor of CYP3A4 in humans, which may result in increased exposures of CYP3A4 substrates. Accordingly, provided herein are methods for administering voxelotor and avoiding or lessening potential drug-drug interactions.

A “strong inhibitor” of a CYP enzyme as used herein is an inhibitor that caused a greater than 5-fold increase in the plasma AUC values or more than 80% decrease in clearance of CYP substrates (not limited to sensitive CYP substrate) in clinical evaluations.

A “moderate inhibitor” is an inhibitor that caused a less than 2-fold but greater than 5-fold increase in the AUC values or 50-80% decrease in clearance of sensitive CYP substrates when the inhibitors was given at the highest approved dose and the shortest dosing interval in clinical evaluations.

As used herein, a “sensitive CYP3A4 substrate” or “sensitive CYP3A4 substrate with a narrow therapeutic index” refers to a modulator of CYP3A4 where small differences in dose or blood concentration of the modulator may lead to dose and blood concentration dependent, serious therapeutic failures or adverse drug reactions. Non-limiting examples of a sensitive CYP3A4 substrate with a narrow therapeutic index include, but are not limited to, aminophylline, cyclobenzaprine, meperidine, temsirolimus, buspirone, rilpivirine, tadalafil, and dasatinib. In some embodiments, the sensitive CYP3A4 substrate with a narrow therapeutic index is alfentanil, cyclosporine, fentanyl, quinidine, sirolimus, or tacrolimus.

Non-limiting examples of a strong CYP3A4 inhibitor include, but are not limited to, VIEKIRA PAK (ombitasavir, partiaprevir, ritonavir and dasabuvir), indinavir/ritonavir, tipranavir, ritonavir, cobicistat, ketoconazole, indinavir, troleandomycin, telaprevir, danoprevir, elvitegravir/ritonavir, saquinavir/ritonavir, lopinavir/ritonavir, itraconazole, voriconazole, mifepristone, mibefradil, LCL161, clarithromycin, posaconazole, telithromycin, grapefruit juice, ceritinib, conivapatan, nefazodone, nelfinavir, saquinavir, ribociclib, idelisib, boceprevir, and atazanavir.

Non-limiting examples of a strong CYP3A4 inducers include, but are not limited to, rifampin, mitotane, avasimibe, rifapentine, apalutamide, ivosidenib, phenytoin, carbamazpeine, enzalutamide, St. John's Wort extract, lumacaftor, and phenobarbital. In some embodiments, methods described herein comprise avoiding co-administration with St. John's wort.

Non-limiting examples of a moderate CYP3A4 inducers are ritonavir and St. John's wort, semagacestat, efavirenz, tipranavir and ritonavir, dabrafenib, lesinurad, bosentan, genistein, thioridazine, rifabutin, lorlatinib, nafcillin, lopinavir, daclatasvir and asunaprevir and beclabuvir, modafinil, PF-06282999, etravirine, elagolix, lersirvine, and teleotristat ethyl.

In some embodiments, voxelotor is not an inhibitor of P-glycoprotein (“P-gp”).

In some embodiments, voxelotor is not an inhibitor of CYP1A2, CYP2C9, CYP2C19, CYP2C8, or CYP2D6. In such embodiments, a patient is administered about 1500 mg of voxelotor per day.

Some embodiments provide for a method of treating sickle cell disease in a patient in need thereof, comprising administering to the patient about 500 mg to about 1500 mg of voxelotor per day, and avoiding co-administration of a strong CYP3A4 inhibitor.

Some embodiments provide for a method of treating sickle cell disease in a patient in need thereof, comprising administering to the patient about 500 mg to about 1500 mg of voxelotor per day, and avoiding co-administration of fluconazole.

Some embodiments provide for a method of treating sickle cell disease in a patient in need thereof, comprising administering to the patient about 500 mg to about 1500 mg of voxelotor per day, and avoiding co-administration of a moderate or a strong CYP3A4 inducer.

Some embodiments provide for a method of treating sickle cell disease in a patient in need thereof, comprising administering to the patient about 600 mg to about 1500 mg of voxelotor per day, and avoiding co-administration of a strong or moderate CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, and avoiding co-administration of a strong CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, and avoiding co-administration of a moderate CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 500 mg to about 1500 mg of voxelotor per day, and avoiding co-administration of a sensitive CYP3A4 substrate with a narrow therapeutic index.

Some embodiments provide for a method of treating sickle cell disease in a patient in need thereof, comprising administering to the patient about 500 mg to about 1500 mg of voxelotor per day, and instructing the patient to avoid co-administration of a strong CYP3A4 inhibitor.

Some embodiments provide for a method of treating sickle cell disease in a patient in need thereof, comprising administering to the patient about 500 mg to about 1500 mg of voxelotor per day, and instructing the patient to avoid co-administration of fluconazole.

Some embodiments provide for a method of treating sickle cell disease in a patient in need thereof, comprising administering to the patient about 500 mg to about 1500 mg of voxelotor per day, and instructing the patient to avoid co-administration of a moderate or a strong CYP3A4 inducer.

Some embodiments provide for a method of treating sickle cell disease in a patient in need thereof, comprising administering to the patient about 600 mg to about 1500 mg of voxelotor per day, and instructing the patient to avoid co-administration of a strong or moderate CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, and instructing the patient to avoid co-administration of a strong CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, and instructing the patient to avoid co-administration of a moderate CYP3A4 inducer, wherein the patient is about 4 years old to less than about 12 years old, and the amount of voxelotor is determined by the patient's body weight.

Some embodiments provide for a method of treating sickle cell disease in a patient in need thereof, comprising administering to the patient about 500 mg to about 1500 mg of voxelotor per day, and instructing the patient to avoid co-administration of a sensitive CYP3A4 substrate with a narrow therapeutic index.

Some embodiments provide for a method of treating sickle cell disease in a patient in need thereof, comprising administering to the patient about 500 mg to about 1500 mg of voxelotor per day, wherein the patient is not administered a strong CYP3A4 inhibitor.

Some embodiments provide for a method of treating sickle cell disease in a patient in need thereof, comprising administering to the patient about 500 mg to about 1500 mg of voxelotor per day, wherein the patient is not administered fluconazole.

Some embodiments provide for a method of treating sickle cell disease in a patient in need thereof, comprising administering to the patient about 500 mg to about 1500 mg of voxelotor per day, wherein the patient is not administered a moderate or a strong CYP3A4 inducer.

Some embodiments provide for a method of treating sickle cell disease in a patient in need thereof, comprising administering to the patient about 600 mg to about 1500 mg of voxelotor per day, wherein the patient is not administered a strong CYP3A4 inducer, the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, wherein the patient is not administered a strong or moderate CYP3A4 inducer, the patient is about 4 years old to less than about 12 years old, and the amount of voxelotor is determined by the patient's body weight.

Some embodiments provide for a method of treating sickle cell disease in a patient in need thereof, comprising administering to the patient about 500 mg to about 1500 mg of voxelotor per day, and wherein the patient is not administered a sensitive CYP3A4 substrate with a narrow therapeutic index.

Some embodiments provide for a method of treating sickle cell disease in a patient in need thereof, comprising administering to the patient about 500 mg to about 1500 mg of voxelotor per day, and wherein the patient is not co-administered a strong CYP3A4 inhibitor.

Some embodiments provide for a method of treating sickle cell disease in a patient in need thereof, comprising administering to the patient about 500 mg to about 1500 mg of voxelotor per day, and wherein the patient is not co-administered fluconazole.

Some embodiments provide for a method of treating sickle cell disease in a patient in need thereof, comprising administering to the patient about 500 mg to about 1500 mg of voxelotor per day, and wherein the patient is not co-administered a moderate or a strong CYP3A4 inducer.

Some embodiments provide for a method of treating sickle cell disease in a patient in need thereof, comprising administering to the patient about 600 mg to about 1500 mg of voxelotor per day, wherein the patient is not co-administered a strong CYP3A4 inducer, the patient is about 4 years old to less than about 12 years old, and the amount of voxelotor is determined by the patient's body weight.

Some embodiments provide for a method of treating sickle cell disease in a patient in need thereof, comprising administering to the patient about 600 mg to about 1500 mg of voxelotor per day, wherein the patient is not co-administered a strong or moderate CYP3A4 inducer, the patient is about 4 years old to less than about 12 years old, and the amount of voxelotor is determined by the patient's body weight.

Some embodiments provide for a method of treating sickle cell disease in a patient in need thereof, comprising administering to the patient about 500 mg to about 1500 mg of voxelotor per day, and wherein the patient is not co-administered a sensitive CYP3A4 substrate with a narrow therapeutic index.

In some embodiments, the patient is administered about 1500 mg of voxelotor per day.

In some embodiments, the sensitive CYP3A4 substrate with a narrow therapeutic index is midazolam, alfentanil, cyclosporine, fentanyl, quinidine, sirolimus, and tacrolimus.

Some embodiments provide for a method of administering voxelotor to a patient in need thereof, comprising first discontinuing administration of a strong CYP3A4 inhibitor to avoid an adverse drug interaction with voxelotor, and then administering to the patient a therapeutically effective amount of voxelotor, wherein the patient has sickle cell disease.

Some embodiments provide for a method of administering voxelotor to a patient in need thereof, comprising first discontinuing administration of fluconazole to avoid an adverse drug interaction with voxelotor, and then administering to the patient a therapeutically effective amount of voxelotor, wherein the patient has sickle cell disease.

Some embodiments provide for a method of administering voxelotor to a patient in need thereof, comprising first discontinuing administration of a moderate or strong CYP3A4 inducer to avoid an adverse drug interaction with voxelotor, and then administering to the patient a therapeutically effective amount of voxelotor, wherein the patient has sickle cell disease.

In some embodiments, the methods comprise first discontinuing administration of a moderate CYP3A4 inducer. In some embodiments, the methods comprise first discontinuing administration of a strong CYP3A4 inducer.

Some embodiments provide for a method of administering voxelotor to a patient in need thereof, comprising first discontinuing administration of a sensitive CYP3A4 substrate with a narrow therapeutic index to avoid an adverse drug interaction with voxelotor, and then administering to the patient a therapeutically effective amount of voxelotor, wherein the patient has sickle cell disease.

In some embodiments, the therapeutically effective amount of voxelotor is about 500 mg to about 1500 mg per day.

In some embodiments, administration of a strong CYP3A4 inhibitor is discontinued concurrently with starting administration of voxelotor. In some embodiments, administration of a strong CYP3A4 inhibitor is discontinued 1, 2, or 3 days prior to starting administration with voxelotor. In some embodiments, administration of a strong CYP3A4 inhibitor is discontinued between 1 day to 7 days prior to starting administration with voxelotor. In some embodiments, administration of a strong CYP3A4 inhibitor is discontinued between 1 day to 14 days prior to starting administration with voxelotor. In some embodiments, administration of a strong CYP3A4 inhibitor is discontinued between 1 day to 21 days prior to starting administration with voxelotor. In some embodiments, administration of a strong CYP3A4 inhibitor is discontinued between 1 day to 1 month prior to starting administration with voxelotor.

In some embodiments, administration of fluconazole is discontinued concurrently with starting administration of voxelotor. In some embodiments, administration of fluconazole is discontinued 1, 2, or 3 days prior to starting administration with voxelotor. In some embodiments, administration of a fluconazole is discontinued between 1 day to 7 days prior to starting administration with voxelotor. In some embodiments, administration of fluconazole is discontinued between 1 day to 14 days prior to starting administration with voxelotor. In some embodiments, administration of fluconazole is discontinued between 1 day to 21 days prior to starting administration with voxelotor. In some embodiments, administration of fluconazole is discontinued between 1 day to 1 month prior to starting administration with voxelotor.

In some embodiments, administration of a moderate CYP3A4 inducer is discontinued concurrently with starting administration of voxelotor. In some embodiments, administration of a moderate CYP3A4 inducer is discontinued 1, 2, or 3 days prior to starting administration with voxelotor. In some embodiments, administration of a moderate CYP3A4 inducer is discontinued between 1 day to 7 days prior to starting administration with voxelotor. In some embodiments, administration of a moderate CYP3A4 inducer is discontinued between 1 day to 14 days prior to starting administration with voxelotor. In some embodiments, administration of a moderate CYP3A4 inducer is discontinued between 1 day to 21 days prior to starting administration with voxelotor. In some embodiments, administration of a moderate CYP3A4 inducer is discontinued between 1 day to 1 month prior to starting administration with voxelotor.

In some embodiments, administration of a strong CYP3A4 inducer is discontinued concurrently with starting administration of voxelotor. In some embodiments, administration of a strong CYP3A4 inducer is discontinued 1, 2, or 3 days prior to starting administration with voxelotor. In some embodiments, administration of a strong CYP3A4 inducer is discontinued between 1 day to 7 days prior to starting administration with voxelotor. In some embodiments, administration of a strong CYP3A4 inducer is discontinued between 1 day to 14 days prior to starting administration with voxelotor. In some embodiments, administration of a strong CYP3A4 inducer is discontinued between 1 day to 21 days prior to starting administration with voxelotor. In some embodiments, administration of a strong CYP3A4 inducer is discontinued between 1 day to 1 month prior to starting administration with voxelotor.

In some embodiments, administration of a strong CYP3A4 inhibitor is discontinued concurrently with starting administration of voxelotor. In some embodiments, administration of a strong CYP3A4 inhibitor is discontinued 1, 2, or 3 days prior to starting administration with voxelotor. In some embodiments, administration of a strong CYP3A4 inhibitor is discontinued between 1 day to 7 days prior to starting administration with voxelotor. In some embodiments, administration of a strong CYP3A4 inhibitor is discontinued between 1 day to 14 days prior to starting administration with voxelotor. In some embodiments, administration of a strong CYP3A4 inhibitor is discontinued between 1 day to 21 days prior to starting administration with voxelotor. In some embodiments, administration of a strong CYP3A4 inhibitor is discontinued between 1 day to 1 month prior to starting administration with voxelotor.

In some embodiments, administration of a sensitive CYP3A4 substrate with a narrow therapeutic index is discontinued concurrently with starting administration of voxelotor. In some embodiments, administration of a sensitive CYP3A4 substrate with a narrow therapeutic index is discontinued 1, 2, or 3 days prior to starting administration with voxelotor. In some embodiments, administration of a sensitive CYP3A4 substrate with a narrow therapeutic index is discontinued between 1 day to 7 days prior to starting administration with voxelotor. In some embodiments, administration of a sensitive CYP3A4 substrate with a narrow therapeutic index is discontinued between 1 day to 14 days prior to starting administration with voxelotor. In some embodiments, administration of a sensitive CYP3A4 substrate with a narrow therapeutic index is discontinued between 1 day to 21 days prior to starting administration with voxelotor. In some embodiments, administration of a sensitive CYP3A4 substrate with a narrow therapeutic index is discontinued between 1 day to 1 month prior to starting administration with voxelotor.

In some embodiments, a method of concurrently administering voxelotor and a sensitive CYP3A4 substrate with a narrow therapeutic index comprises administering to a patient a therapeutically effective amount of voxelotor and a dosage of a sensitive CYP3A4 substrate with a narrow therapeutic index that is decreased relative to a patient taking a sensitive CYP3A4 substrate with a narrow therapeutic index.

In some embodiments, a method of concurrently administering voxelotor and a strong or moderate CYP3A4 inducer comprises administering to a patient a therapeutically effective amount of a strong or moderate CYP3A4 inducer and a dosage of voxelotor that is increased relative to a patient not taking a strong or moderate CYP3A4 inducer.

In some embodiments, the dosage of voxelotor that is increased is 2,500 mg per day. In some embodiments, the dosage of voxelotor that is increased is 2000 mg per day.

Some embodiments provide for methods of concurrently administering voxelotor and a strong CYP3A4 inducer comprising administering to a patient a therapeutically effective amount of a strong CYP3A4 inducer and 2000 mg voxelotor orally once daily, wherein the patient is 12 years of age or older.

Some embodiments provide for methods of concurrently administering voxelotor and a strong CYP3A4 inducer comprising administering to a patient a therapeutically effective amount of a strong CYP3A4 inducer and 2500 mg voxelotor orally once daily, wherein the patient is 12 years of age or older.

Some embodiments provide for methods of concurrency administering voxelotor and a moderate CYP3A4 inducer comprising administering to a patient a therapeutically effective amount of a moderate CYP3A4 inducer and 2000 mg voxelotor orally once daily, wherein tie patient is 12 years of age or older.

Some embodiments provide for methods of concurrently administering voxelotor and a strong CYP3A4 inducer comprising administering to a patient a therapeutically effective amount of a strong CYP3A4 inducer and 900 mg to 2100 ng voxelotor orally once daily, wherein the patient is about 4 years old to less than about 12 years old, and the amount of voxelotor is determined by the patient's body weight.

In some embodiments, the patient's body weight is about 40 kg or greater (e.g., greater than about 40 kg), and the patient is administered 2000 mg of voxelotor per day. In some embodiments, the patient's body weight is about 40 kg or greater (e.g., greater than about 40 kg), and the patient is administered 2100 mg of voxelotor per day. In some embodiments, the patient's body weight is about 20 kg to less than about 40 kg, and the patient is administered 1200 mg of voxelotor per day. In some embodiments, the patient's body weight is about 10 kg to less than about 20 kg, and the patient is administered 900 mg of voxelotor per day.

Some embodiments provide for methods of concurrently administering voxelotor and a strong CYP3A4 inducer comprising administering to a patient a therapeutically effective amount of a strong CYP3A4 inducer and 2000 mg voxelotor orally once daily, wherein the patient is about 4 years old to less than about 12 years old, and the patient's body weight is about 40 kg or greater (e.g., greater than about 40 kg).

Some embodiments provide for methods of concurrently administering voxelotor and a strong CYP3A4 inducer comprising administering to a patient a therapeutically effective amount of a strong CYP3A4 inducer and 2100 mg voxelotor orally once daily, wherein the patient is about 4 years old to less than about 12 years old, and the patient's body weight is about 40 kg or greater (e.g., greater than about 40 kg).

Some embodiments provide for methods of concurrently administering voxelotor and a strong CYP3A4 inducer comprising administering to a patient a therapeutically effective amount of a strong CYP3A4 inducer and 1200 mg voxelotor orally once daily, wherein the patient is about 4 years old to less than about 12 years old, and the patient's body weight is about 20 kg to less than about 40 kg.

Some embodiments provide for methods of concurrently administering voxelotor and a strong CYP3A4 inducer comprising administering to a patient a therapeutically effective amount of a strong CYP3A4 inducer and 900 ng voxelotor orally once daily, wherein the patient is about 4 years old to less than about 12 years old, and the patient's body weight is about 10 kg to less than about 20 kg.

Some embodiments provide for methods of concurrently administering voxelotor and a strong CYP3A4 inducer comprising administering to a patient a therapeutically effective amount of a strong CYP3A4 inducer and 900 mg to 2500 ng voxelotor orally once daily, wherein the patient is about 4 years old to less than about 12 years old, and the amount of voxelotor is determined by the patient's body weight.

In some embodiments, the patient's body weight is about 40 kg or greater, and the patient is administered 2500 mg of voxelotor per day. In some embodiments, the patient's body weight is about 40 kg or greater, and the patient is administered 2400 mg of voxelotor per day. In some embodiments, the patient's body weight is about 20 kg to less than about 40 kg, and the patient is administered 1500 mg of voxelotor per day. In some embodiments, the patient's body weight is about 10 kg to less than about 20 kg, and the patient is administered 900 mg of voxelotor per day.

Some embodiments provide for methods of concurrently administering voxelotor and a strong CYP3A4 inducer comprising administering to a patient a therapeutically effective amount of a strong CYP3A4 inducer and 2500 mg voxelotor orally once daily, wherein the patient is about 4 years old to less than about 12 years old, and the patient's body weight is about 40 kg or greater.

Some embodiments provide for methods of concurrently administering voxelotor and a strong CYP3A4 inducer comprising administering to a patient a therapeutically effective amount of a strong CYP3A4 inducer and 2400 mg voxelotor orally once daily, wherein the patient is about 4 years old to less than about 12 years old, and the patient's body weight is about 40 kg or greater.

Some embodiments provide for methods of concurrently administering voxelotor and a strong CYP3A4 inducer comprising administering to a patient a therapeutically effective amount of a strong CYP3A4 inducer and 1500 mg voxelotor orally once daily, wherein the patient is about 4 years old to less than about 12 years old, and the patient's body weight is about 20 kg to less than about 40 kg.

Some embodiments provide for methods of concurrently administering voxelotor and a moderate CYP3A4 inducer comprising administering to a patient a therapeutically effective amount of a moderate CYP3A4 inducer and 900 mg to 2100 mg voxelotor orally once daily, wherein the patient is about 4 years old to less than about 12 years old, and the amount of voxelotor is determined by the patient's body weight.

In some embodiments, the patient's body weight is about 40 kg or greater, and the patient is administered 2000 mg of voxelotor per day. In some embodiments, the patient's body weight is about 40 kg or greater, and the patient is administered 2100 mg of voxelotor per day. In some embodiments, the patient's body weight is about 20 kg to less than about 40 kg, and the patient is administered 1200 mg of voxelotor per day. In some embodiments, the patient's body weight is about 10 kg to less than about 20 kg, and the patient is administered 900 mg of voxelotor per day.

Some embodiments provide for methods of concurrently administering voxelotor and a moderate CYP3A4 inducer comprising administering to a patient a therapeutically effective amount of a moderate CYP3A4 inducer and 2000 ng voxelotor orally once daily, wherein the patient is about 4 years old to less than about 12 years old, and the patient's body weight is about 40 kg or greater.

Some embodiments provide for methods of concurrently administering voxelotor and a moderate CYP3A4 inducer comprising administering to a patient a therapeutically effective amount of a moderate CYP3A4 inducer and 2100 mg voxelotor orally once daily, wherein the patient is about 4 years old to less than about 12 years old, and the patient's body weight is about 40 kg or greater.

Some embodiments provide for methods of concurrently administering voxelotor and a moderate CYP3A4 inducer comprising administering to a patient a therapeutically effective amount of a moderate CYP3A4 inducer and 1200 mg voxelotor orally once daily, wherein the patient is about 4 years old to less than about 12 years old, and the patient's body weight is about 20 kg to less than about 40 kg.

Some embodiments provide for methods of concurrently administering voxelotor and a moderate CYP3A4 inducer comprising administering to a patient a therapeutically effective amount of a moderate CYP3A4 inducer and 900 mg voxelotor orally once daily, wherein the patient is about 4 years old to less than about 12 years old, and the patient's body weight is about 10 kg to less than about 20 kg.

In some embodiments, a method of concurrently administering voxelotor and fluconazole or a strong CYP3A4 inhibitor comprises administering to a patient a therapeutically effective amount of fluconazole or a strong CYP3A4 inhibitor and a dosage of voxelotor that is decreased relative to a patient not takin fluconazole or a strong CYP3A4 inhibitor.

In some embodiments, the dose of voxelotor that is decreased is about 1,000 mg per day. In some embodiments, the dose of voxelotor that is decreased is about 500 mg per day.

In some embodiments, the patient is already being administered a sensitive CYP3A4 substrate with a narrow therapeutic index, moderate CYP3A4 inducer, strong CYP3A4 inducer, fluconazole, or strong CYP3A4 inhibitor. In some embodiments, the patient is already being administered voxelotor.

In some embodiments, the dosage of voxelotor is increased prior to administration of a moderate or strong CYP3A4 inducer.

In some embodiments, the dosage of voxelotor is decreased prior to administration of a strong CYP3A4 inhibitor or fluconazole.

Also provided herein are methods of administering voxelotor to a patient in need thereof, comprising administering to the patient a therapeutically effective amount of voxelotor, and advising the patient one or more of the following:
(a) advising the patient that a sensitive CYP3A4 substrate with a narrow therapeutic index, moderate CYP3A4 inducer, strong CYP3A4 inducer, fluconazole, or strong CYP3A4 inhibitor should be avoided or discontinued;
(b) advising the patient that co-administration of voxelotor with a sensitive CYP3A4 substrate with a narrow therapeutic index, moderate CYP3A4 inducer, strong CYP3A4 inducer, fluconazole, or strong CYP3A4 inhibitor can alter the therapeutic effect of voxelotor;
(c) advising the patient that co-administration of voxelotor and a moderate CYP3A4 inducer or strong CYP3A4 inducer can decrease voxelotor plasma concentrations and may lead to reduced efficacy;
(d) advising the patient that co-administration of voxelotor and a strong CYP3A4 inhibitor can increase voxelotor plasma concentrations and may lead to increased toxicity;
(e) advising the patient that co-administration of voxelotor with a sensitive CYP3A4 substrate with a narrow therapeutic index can increase the systemic exposure of the sensitive CYP3A4 substrate with a narrow therapeutic index;
(f) advising the patient to replace a strong CYP3A4 inhibitor or fluconazole with an alternative drug;
(g) if co-administration of a strong CYP3A4 inhibitor or fluconazole is unavoidable, decreasing the dose of voxelotor to 1000 mg per day;
(h) if co-administration of a moderate CYP3A4 inducer or strong CYP3A4 inducer is unavoidable, increasing the dose of voxelotor to 2500 mg per day;
(i) if co-administration of a CYP3A4 inducer is unavoidable and the patient is about 12 years old or older, increasing the dose of voxelotor to 2000 mg per day;
(j) if co-administration of a CYP3A4 inducer is unavoidable and the patient is about 4 years old to less than about 12 years old, increasing the dose of voxelotor to 900 mg to 2100 ng voxelotor mg per day depending on the patient's body weight;
(k) if co-administration of a strong CYP3A4 inducer is unavoidable and the patient is about 12 years old or older, increasing the dose of voxelotor to 2500 mg per day;
(l) if co-administration of a moderate CYP3A4 inducer is unavoidable and the patient is about 12 years old or older, increasing the dose of voxelotor to 2000 mg per day;
(m) if co-administration of a strong CYP3A4 inducer is unavoidable and the patient is about 4 years old to less than about 12 years old, increasing the dose of voxelotor to 900 ng to 2500 mg voxelotor mg per day depending on the patient's body weight;
(n) if co-administration of a moderate CYP3A4 inducer is unavoidable and the patient is about 4 years old to less than about 12 years old, increasing the dose of voxelotor to 900 mg to 2100 mg voxelotor mg per day depending on the patient's body weight; or
(o) if co-administration of a sensitive CYP3A4 substrate with a narrow therapeutic index is unavoidable, decreasing the dose of the sensitive CYP3A4 substrate with a narrow therapeutic index.

### Dosage Forms of Voxelotor

Voxelotor may be administered in any suitable dosage form, including an oral dosage form. In some embodiments, voxelotor is administered to the patient in an oral formulation. In some embodiments, the oral formulation is tablet.

In some embodiments, voxelotor is administered as a capsule. Capsules of voxelotor are described in U.S. Patent Publication No. 2017/0157101, which is hereby incorporated by reference in its entirety.

In some embodiments, voxelotor is administered as a tablet. Tablets and dispersible tablets of voxelotor are described in U.S. Patent Publication No. US 2018/0125789, which is hereby incorporated by reference in its entirety.

In some embodiments, the tablets described herein comprise voxelotor as a substantially pure crystalline ansolvate form characterized by at least two X-ray powder diffraction peaks (Cu Kα radiation) selected from 13.37°, 14.37°, 19.950 and 23.92° 2θ (each ±0.2 °2θ). In some embodiments, the tablets described herein consists essentially of voxelotor as a crystalline ansolvate form characterized by at least two X-ray powder diffraction peaks (Cu Kα radiation) selected from 13.37°, 14.37°, 19.950 and 23.92° 2θ (each ±0.2 °2θ). In some embodiments, the tablets described herein comprise a crystalline ansolvate form of voxelotor characterized by X-ray powder diffraction peaks (Cu Kα radiation) at 13.37°, 14.37°, 19.950 and 23.92° 2θ (each ±0.2 °2θ).

The tablets described herein comprise voxelotor (e.g., Form I, Form II, or Form N) at an amount of, or at an amount of about, 55%, 56%, 57%, 58%, 59.1%, 59.2%, 59.3%, 59.4%, 59.5%, 59.6%, 59.7%, 59.8%, 59.9%, 60%, 61%, 62%, 63%, 64%, 65% w/w, or a range between any two of these values, wherein the percentage by weight is relative to the total weight of the tablet. In some embodiments, the tablet comprises, or comprises about, 50% to about 70% w/w of voxelotor (e.g., Form II). In some embodiments, the tablet comprises, or comprises about 60% w/w of voxelotor (e.g., Form II).

The tablets described herein comprise about 300 mg to about 1500 mg or about 300 mg to about 900 mg of voxelotor (e.g., Form I, Form II, or Form N). The tablets described herein comprise about 100 mg to about 600 mg of voxelotor (e.g., Form I, Form II, or Form N). In some embodiments, the tablets described herein comprise voxelotor (e.g., Form I, Form II, or Form N) in an amount of, or in an amount of about 500 mg. In some embodiments, the tablets described herein comprise voxelotor (e.g., Form I, Form II, or Form N) in an amount of, or in an amount of about 300 mg.

In some embodiments, methods described herein comprise administering one to ten tablets, wherein each tablet comprises 500 mg voxelotor. In some embodiments, methods described herein comprise administering one to ten tablets, wherein each tablet comprises 300 mg voxelotor.

In some embodiments, methods described herein comprise administering one to eight tablets, wherein each tablet comprises 500 mg voxelotor. In some embodiments, methods described herein comprise administering one to eight tablets, wherein each tablet comprises 300 mg voxelotor.

In some embodiments, methods described herein comprise administering one to seven tablets, wherein each tablet comprises 500 mg voxelotor. In some embodiments, methods described herein comprise administering one to seven tablets, wherein each tablet comprises 300 mg voxelotor.

In some embodiments, methods described herein comprise administering four tablets, wherein each tablet comprises 500 mg voxelotor. In some embodiments, methods described herein comprise administering three tablets, wherein each tablet comprises 500 mg voxelotor. In some embodiments, methods described herein comprise administering two tablets, wherein each tablet comprises 500 mg voxelotor.

In some embodiments, methods described herein comprise administering eight tablets, wherein each tablet comprises 300 mg voxelotor. In some embodiments, methods described herein comprise administering seven tablets, wherein each tablet comprises 300 mg voxelotor. In some embodiments, methods described herein comprise administering six tablets, wherein each tablet comprises 300 mg voxelotor. In some embodiments, methods described herein comprise administering five tablets, wherein each tablet comprises 300 mg voxelotor. In some embodiments, methods described herein comprise administering four tablets, wherein each tablet comprises 300 mg voxelotor. In some embodiments, methods described herein comprise administering three tablets, wherein each tablet comprises 300 mg voxelotor. In some embodiments, methods described herein comprise administering two tablets, wherein each tablet comprises 300 mg voxelotor. In some embodiments, methods described herein comprise administering one tablet, wherein the tablet comprises 300 mg voxelotor.

In some embodiments, methods described herein comprise administering four tablets, wherein each tablet comprises 500 mg voxelotor. In some embodiments, methods described herein comprise administering seven tablets, wherein each tablet comprises 300 mg voxelotor. In some embodiments, methods described herein comprise administering eight tablets, wherein each tablet comprises 300 mg voxelotor.

The tablets disclosed herein comprise excipients such as a pharmaceutically acceptable filler (also known as diluent), disintegrant, lubricant, surfactant (also known as wetting agent), glidant, and binder.

In some embodiments, the tablets described herein comprise microcrystalline cellulose (MCC). In some embodiments, the MCC is present in the tablet at, or at about, 30%, 31%, 32%, 33%, 34%, 35%, 36%, 37%, 38%, 39%, 40%, or a range between any two of these values, w/w. In some embodiments, the MCC is present in the tablet at about 30% to about 40% w/w. In some embodiments, the MCC is present in the tablet at, or at about, 35% w/w.

The tablets described herein comprise, in some embodiments, one or more disintegrants. Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like.

In some embodiments, the disintegrant is croscarmellose sodium. The tablets comprise, or comprise about, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5% 1.6%, 1.7%, 1.8%, 1.9%, or 2% or a range between any two of these values, w/w of one or more disintegrants. In some embodiments, the tablets comprise about 0.25% to about 3% w/w of a disintegrant. In some embodiments, the tablets comprise about 0.25% to about 3% w/w of croscarmellose sodium. In some embodiments, the tablets comprise about 1.25% w/w of croscarmellose sodium.

The tablets described herein comprise one or more surfactants (also known as wetting agents). In some embodiments, the tablets comprise sodium lauryl sulfate as a surfactant. In some embodiments, the surfactant can be present in the tablets disclosed herein at an amount of, or at an amount of about, 0.1%, 0.5%, 1%, 1.1%, 1.2%, 1.3%, 1.4%, 1.45%, 1.5%, 1.55%, 1.6%, 1.65%, 1.7%, 1.75%, 1.8%, 1.9%, 2.0%, 2.5%, 3%, 4%, 5%, or a range between any two of these values, w/w. In some embodiments, the tablets do not comprise a surfactant. In some embodiments, the tablets comprise about 0.5% to about 2.5% w/w of sodium lauryl sulfate. In some embodiments, the tablets comprise sodium lauryl sulfate at an amount of about 1.5% w/w.

The tablets described herein comprise one or more lubricants. Exemplary lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. In some embodiments, the lubricant can be present in the tablets at an amount of, or at an amount of about, 0.25%, 0.5%, 0.75%, 1%, 1.25%, 1.5%, 1.75%, 2%, 2.5%, 2.75%, 3%, 4%, 5%, or a range between any two of these values, w/w. In some embodiments, the lubricant is magnesium stearate. In some embodiments, magnesium stearate is present in the tablets in the amount of from about 1% to about 5% w/w. In some embodiments, magnesium stearate is present in the tablets in the amount of about 2% w/w.

The tablets described herein comprise one or more binders. Exemplary binding agents and adhesives include, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; and the like.

The tablets described herein comprise one or more glidants. Exemplary glidants include, colloidal silicon dioxide, starch, powdered cellulose, sodium lauryl sulfate, magnesium trisilicate and metallic stearates. In some embodiments, the glidant is colloidal silicon dioxide. In some embodiments, the tablet comprises about 0.1% to about 5% by weight of colloidal silicon. In some embodiments, the tablet comprises about 0.5% to about 1% by weight of colloidal silicon dioxide. In some embodiments, the tablet comprises about 0.75% by weight of colloidal silicon dioxide.

Other excipients such as colorants (coloring agents), coating polymers, flavors (flavoring agents), and sweeteners are known in the pharmaceutical art and can be used in the tablets disclosed herein. Non-limiting examples of a sweetener are sucrose, xylitol, maltitol, mannitol, sorbitol, sucralose, sodium saccharin, acesulfame potassium, aspartame, and others known to those of skill in the art. In some embodiments, the coloring agent is iron oxide yellow.

In some embodiments, a tablet comprises about 50% to about 70% by weight voxelotor; about 30% to about 40% by weight of microcrystalline cellulose; about 0.25% to about 3% by weight of croscarmellose sodium; about 1% to about 5% by weight of magnesium stearate; about 0.5% to about 2.5% of sodium lauryl sulfate; and about 0.25% to about 5% by weight of colloidal silicon dioxide; wherein the percentage by weight is relative to the total weight of the tablet. In some embodiments, a tablet is a dispersible tablet. In some embodiments, a dispersible tablet comprises 60%±5% by weight voxelotor; 20% to 35% by weight of microcrystalline cellulose; 1% to 2% by weight of croscarmellose sodium; less than 2% by weight of colloidal silicon dioxide; 1% to about 5% by weight of magnesium stearate; a sweetener; a flavoring agent; and a coloring agent; wherein the percentage by weight is relative to the total weight of the tablet.

In some embodiments, a dispersible tablet comprises about 50% to about 70% by weight voxelotor; about 20% to about 35% by weight of microcrystalline cellulose; about 1% to about 2% by weight of croscarmellose sodium; less than about 2% by weight of colloidal silicon dioxide; about 1% to about 5% by weight of magnesium stearate; optionally about 1% to about 3% by weight of sweetener; optionally about 0.2% to about 1.5% by weight of flavoring agent; and optionally about 0.1% to about 1% by weight of coloring agent wherein the percentage by weight is relative to the total weight of the tablet.

In some embodiments, tablets described herein include a coating surrounding the core described herein comprising voxelotor. Tablets can be coated using formulations known in the art, such as for example, excipients such as talc, polyvinyl alcohol, and PEG (e.g., PEG 4000 and PEG 6000). In some embodiments, the coating polymer can be hydroxypropyl methylcellulose (HPMC). When coated, tablets comprise a core that is coated with a nonfunctional film or a release-modifying or enteric coating.

In some embodiments, voxelotor is administered as an oral suspension. In some embodiments, a tablet of voxelotor is dispersed in a liquid before administration. In some embodiments, the liquid is a clear liquid. In some embodiments, the clear liquid is water, juice (such as apple juice), flavored sports drink, soda (for example, a clear soda), or flavored electrolyte drink. In some embodiments, the clear liquid is water, soda (for example, a clear soda), juice (such as apple juice), a clear electrolyte drink, a clear flavored drink, or a clear sports drink. In some embodiments, the clear liquid is water or a clear soda. In some embodiments, the tablet of voxelotor is dispersed within 1 to 5 minutes. In some embodiments, the tablet does not completely dissolve, thereby providing a residue; in such embodiments, the residue may be resuspended in a liquid before administration.

In some embodiments, one tablet of voxelotor is dispersed in 5 mL of liquid. In some embodiments, two tablets of voxelotor are dispersed in 10 mL of liquid. In some embodiments, three tablets of voxelotor are dispersed in 15 mL of liquid. In some embodiments, four tablets of voxelotor are dispersed in 20 mL of liquid. In some embodiments, four tablets of voxelotor are dispersed in 20 mL of liquid. In some embodiments, five tablets of voxelotor are dispersed in 25 mL of liquid. In some embodiments, six tablets of voxelotor are dispersed in 30 mL of liquid. In some embodiments, seven tablets of voxelotor are dispersed in 35 mL of liquid. In some embodiments, eight tablets of voxelotor are dispersed in 40 mL of liquid. In such embodiments, the tablets dispersed in the liquids comprise 300 mg of voxelotor.

It is understood that modifications which do not substantially affect the activity of the various embodiments of this disclosure are also included within the definition of the disclosure provided herein. Accordingly, the following examples are intended to illustrate but not limit the present disclosure.

### EXAMPLES

### Example 1: Pediatric Patients with SCD Aged 4 to 11 Years

Patients and treatment: Patients with SCD were screened at 15 study sites in the United States, Lebanon, and the United Kingdom. Key inclusion criteria included patients with SCD (homozygous sickle cell disease [HbSS] or HbSβ⁰ genotypes), aged 4 to 11 years, and with hemoglobin (Hb) concentrations ≤10.5 g/dL at baseline. Concomitant hydroxyurea was allowed if the dose was stable for ≥3 months at enrollment, with no anticipated need for dose adjustment during the study. There was no eligibility requirement regarding prior VOCs. Patients who were receiving chronic red blood cell transfusion therapy, had received a transfusion in the past 30 days, or had been hospitalized for a VOC, acute chest syndrome, splenic sequestration crisis, or dactylitis within 14 days before providing informed consent were excluded.

All patients received dispersible voxelotor tablets. Patients aged 4 to 11 years received once-daily voxelotor weight-based-dosing (Table 1).

TABLE 1

| | |
|---|---|
| Weight | Voxelotor dose |

| | | | |
|---|---|---|---|
| 10 to <20 | kg | 600 | mg |
| 20 to <40 | kg | 900 | mg |
| ≥40 | kg | 1500 | mg |

Dose modification (reduction or interruption) or discontinuation of study drug was recommended to manage tolerability issues related to voxelotor treatment. Temporary dose modification was permitted for management of suspected drug-related adverse events. The original dose was resumed after resolution of the adverse event, at the discretion of the investigator.

Endpoints and assessments: Patients were assessed at the screening visit, day 1, and weeks 2, 4, 8, 12, 16, 20, 24, 36, and 48. Efficacy endpoints evaluated in the current analyses included the percentage of patients who had a Hb response, which was defined as an increase from baseline of >1.0 g/dL at week 24; the change in Hb level from baseline to week 24; and the percent change in laboratory markers associated with hemolysis, including indirect bilirubin, reticulocytes, and lactate dehydrogenase (LDH), from baseline to week 24. The safety assessment included treatment-emergent adverse events (TEAEs) and adverse events that led to treatment modification or discontinuation.

Population PK modeling was used to determine the whole-blood and plasma PK parameters of voxelotor in the study population. The PK parameters included maximum whole blood or plasma concentration (Cₘₐₓ), area under the concentration-time curve (AUC), terminal elimination half-life (t_{1/2}), and percent Hb occupancy.

Statistical analyses: All efficacy and safety assessments were summarized with descriptive statistics. Population PK analyses using nonlinear mixed-effects modeling were performed to characterize voxelotor PK in plasma and whole blood.

### Results

Patients: 45 patients aged 4 to 11 years were enrolled in part C the clinical trial. Among these patients, 27 had completed the study, of whom 21 rolled over to the open-label extension study; 10 had discontinued early; and 8 were receiving ongoing treatment. Baseline demographics and clinical characteristics are shown in Table 2.

TABLE 2
Baseline Characteristics of Patients Aged 4 to 11 Years

| | 4 to 11 years | |
|---|---|---|
| Age group | (N = 45) | |
| Age at screening, median (range), years | 7.0 | (4-11) |
| Female, n (%) | 23 | (51.1) |
| Weight at screening, median (range), kg | 24.0 | (12-41) |
| Baseline Hb, g/dL^{a} | | |
| Mean (SD) | 8.6 | (1.01) |
| Range | 6.1-10.5 | |
| Baseline % reticulocyte count^{a} | | |
| Mean (SD) | 10.4 | (4.45) |
| Range | 2.1-19.5 | |
| Baseline HbF, %^{a} | | |
| Mean (SD) | 17.7 | (7.86) |
| Range | 2.3-38.4 | |
| SCD genotype, n (%) | | |
| HbSS | 43 | (95.6) |
| HbSβ⁰ | 2 | (4.4) |
| Current hydroxyurea/hydroxycarbamide use, n (%) | | |
| Yes | 38 | (84.4) |
| No | 7 | (15.6) |
| No VOCs in the previous 12 months, n (%) | 21 | (46.7) |
| ≥1 VOC in the previous 12 months, n (%) | 24 | (53.3) |

Hb, hemoglobin;
HbF, fetal hemoglobin;
HbSβ⁰, sickle beta zero thalassemia;
HbSS, homozygous sickle cell disease;
SCD, sickle cell disease;
VOC, vaso-occlusive crisis
^{a}Baseline is defined as the average of all values before the first dose.

At baseline, the mean (SD) Hb was 8.6 g/dL (1.01 g/dL), and the mean (SD) percent reticulocyte count was 10.4% (4.45%). Baseline mean fetal Hb percentage was 17.7%, consistent with 84.4% of patients receiving concomitant hydroxyurea at a stable dose.

Efficacy: An increase in mean Hb levels was observed as early as 2 weeks after voxelotor initiation and was maintained through week 24 (data not shown). The mean (SD) change in Hb from baseline at week 24 (n=34) was 1.0 g/dL (1.21 g/dL); 47.1% (95% CI, 29.8%-64.9%) of patients achieved a Hb response, defined for this study as an increase in Hb of >1.0 g/dL from baseline to week 24 (data not shown). At week 24, 35% (n= 12/34) and 21% (n= 7/34) of patients had a >1.5 g/dL increase and a >2.0 g/dL increase in Hb from baseline, respectively. Overall, 82% of patients had an increase in Hb of any magnitude from baseline at week 24. Additionally, concurrent reductions in hemolytic markers were observed with voxelotor therapy. The mean (range) percent change from baseline at week 24 was −38.6% (−76.0% to 40.0%; n=28) for indirect bilirubin, −3.25% (−95.0% to 110.3%; n=31) for percent reticulocyte count, and −2.6% (−36.6% to 44.2%; n=32) for lactate dehydrogenase.

Pharmacokinetics: Voxelotor PK parameter estimates for whole blood and plasma are summarized in Table 3.

TABLE 3
PK Parameters of Voxelotor in Patients
Aged 4 to 11 Years in Part C

| | PK parameter |
|---|---|
| | 4-11 years |
| | Voxelotor 600/900/1500 mg^{a} |
| Whole blood PK | (n = 38) |
| AUC_{0-24 h} (μg · h/mL)^{b} | 3257 (31.2) |
| Cₘₐₓ (μ/mL)^{b} | 148.3 (30.2) |
| t_{1/2} (h)^{b} | 27.17 (28.3) |
| % Hb occupancy^{b} | 26.16 (32.2) |
| % of patients with ≥20% Hb occupancy | 76.3 |

AUC_{0-24 h}, area under the concentration-time curve from 0 to 24 hours; Cₘₐₓ, maximum whole blood or plasma concentration; CV, coefficient of variation; Hb, hemoglobin; PK, pharmacokinetics; t_{1/2′} terminal elimination half-life.
^{a}Weight-based dosing for pediatric patients aged 4 to 11 years.
^{b}Geometric mean (% CV).

A target therapeutic range of 20% to 30% Hb occupancy was set. This target is based on the observation that individuals who are compound heterozygous for HbS and a deletional form of hereditary persistence of fetal Hb and who maintain a pancellular fetal Hb distribution of 20% to 30% generally do not exhibit clinical manifestations of SCD. Approximately 76% of patients aged 4 to 11 years achieved Hb occupancy ≥20% with a weight-based dose of voxelotor.

Safety: Safety analyses included all patients who received at least 1 dose of the study drug (Table 4).

TABLE 4
Safety Assessments at Week 24
Drug-related AEs (preferred term) reported for ≥2 patients

| | 4-11 years |
|---|---|
| Adverse events,^{a} n (%) | (n = 45) |
| Any drug-related TEAE | 22 (48.9) |
| Diarrhea | 5 (11.1) |
| Rash^{b} | 5 (11.1) |
| Vomiting | 5 (11.1) |
| Abdominal pain^{c} | 4 (8.9) |
| Transaminases increased^{d} | 4 (8.9) |
| Headache | 2 (4.4) |
| Hypersplenism | 2 (4.4) |
| Pyrexia | 2 (4.4) |

AE, adverse event; ALT, alanine aminotransferase; AST, aspartate aminotransferase; CTCAE, Common Terminology Criteria for Adverse Events; MedDRA, Medical Dictionary for Regulatory Activities; NCI, National Cancer Institute; SCD, sickle cell disease; TEAE, treatment-emergent adverse event.
^{a}AEs were coded using MedDRA version 23.0. NCI-CTCAE version 4.03 was used to determine grade. Patients may be counted in more than 1 row.
^{b}Includes the following preferred terms: rash, rash maculopapular, and rash papular.
^{c}Includes the following preferred terms: abdominal pain and abdominal pain upper.
^{d}Includes the following preferred terms: ALT increased, AST increased, transaminases increased.

The median exposure was 43.3 weeks. Weight-based dosing of voxelotor was well tolerated in this patient population; 49% (n=22) of patients experienced 1 or more TEAEs considered to be related to voxelotor as assessed by the investigator. The most common non-SCD related TEAEs (reported in ≥20% of patients) were pyrexia (35.6%), vomiting (33.3%), and rash (20%). The most commonly reported drug-related TEAEs (reported in ≥10% of patients) were diarrhea (11%), rash (11%), and vomiting (11%); of these, most were grade 1; grade 2 rash was reported in 2 instances, and no events of grade 3 or higher severity were observed. Only 1 temporary dose reduction occurred in 1 patient who had a drug-related TEAE of grade 2 rash that led to an initial dose interruption for 5 days, followed by a dose reduction for 8 doses, and ultimately restoration of the original dose, with no further incidence of rash and subsequent patient enrollment into the open-label extension study. Three additional patients had temporary dose interruptions due to drug-related TEAEs, 1 patient with a grade 2 allergic reaction, 1 patient with a grade 2 ALT increase and a grade 2 AST increase, and 1 patient with a grade 3 bilirubin increase and a grade 3 liver transaminase increase. In both patients with elevated liver enzyme TEAEs, the events resolved after the dose interruption with no further incidence, and both patients enrolled into the open-label extension study for continued voxelotor dosing. The patient with a temporary dose interruption due to a grade 2 allergic reaction recovered from this event but discontinued voxelotor due to multiple discrete drug-related adverse events of grade 2 allergic edema, grade 2 pyrexia, and grade 2 sickle cell anemia with crisis. Nonetheless, overall drug discontinuation due to TEAEs was low. A total of 4 patients discontinued voxelotor due to adverse events. Of these, 2 discontinuations were due to drug-related adverse events, including the patient described above and a second patient who discontinued due to a grade 1 TEAE of decreased appetite. The remaining 2 adverse events leading to discontinuation were unrelated to voxelotor treatment, as assessed by investigators: one patient discontinued due to grade 3 osteonecrosis and grade 3 anemia, and a second patient discontinued due to grade 2 non-cardiac chest pain. There were no deaths reported.

These results from part C of the clinical trial show that the efficacy of voxelotor in patients with SCD aged 4 to 11 years is consistent with that observed in adult and pediatric (aged ≥12 years) patients in a phase 3 clinical trial. Hb response (defined in this study as >1.0 g/dL increase from baseline) was achieved by 54.5% of patients as early as week 2 and maintained through week 24 in 47.1% of patients. In addition, 35% and 21% of patients achieved Hb increases >1.5 g/dL and >2.0 g/dL, respectively. Overall, 82% patients had an increase in Hb from baseline at week 24. The mean increase in Hb from baseline at week 24 was 1.0 g/dL. Furthermore, at week 24, concurrent improvements in hemolytic markers were also observed. These results are comparable to findings from the phase 3 clinical trial, where 51% of adult and pediatric (aged 12 years) patients treated with voxelotor 1500 mg demonstrated a Hb response, and the mean change in Hb from baseline at week 24 was 1.1 g/dL

The mean Hb occupancy achieved with voxelotor weight-based dosing with dispersible tablets was 26%, which is within the target therapeutic range of 20% to 30% Hb occupancy.

The safety profile of voxelotor in patients 4 to 11 years of age was consistent with the existing safety profile in adult and pediatric (aged 12 years) patients. Approximately 49% of patients reported at least 1 drug-related TEAE. The most common non-SCD related TEAEs (reported in 20% of patients), were pyrexia, vomiting, and rash. The most commonly reported drug-related TEAEs (reported in 10% of patients) were diarrhea, vomiting, and rash, which were mostly grade 1 in severity. Temporary dose modifications allowed per the protocol to manage TEAEs were low, with only 1 patient requiring a temporary dose reduction due to a drug-related TEAE of grade 2 rash, and only 3 patients requiring temporary dose interruptions due to drug-related TEAEs. A total of 4 out of 45 patients discontinued voxelotor treatment due to a TEAE, 2 due to drug-related adverse events and 2 due to adverse events unrelated to voxelotor. No new safety signals were identified.

### Example 2: Characterization of Pharmacokinetics and Safety of a Single Oral Dose of Voxelotor in Subjects with Hepatic Impairment

A clinical trial study of a single oral dose of voxelotor administered in pediatric subjects with mild (Child-Pugh A; score 5 to 6 points), moderate (Child-Pugh B; score 7 to 9 points), or severe (Child-Pugh C; score 10 to 15 points) hepatic impairment and pediatric subjects with normal hepatic function is performed to assess the effects of mild, moderate, or severe hepatic impairment on the pharmacokinetics (PK) of a single oral dose of voxelotor. Routine safety assessments (AE monitoring, vital signs, physical examinations, clinical laboratory tests, and ECGs) are performed.

The group demographics between the normal hepatic function group and the mild, moderate, and severe hepatic impairment groups are similar (i.e., with respect to age [±10 years], gender, and BMI [±10%]). The screening window is 28 days in duration.

Subjects with mild and moderate hepatic impairment are enrolled first. Once at least 4 subjects in each of these 2 groups complete the clinical portion of the study (through Day 5 PK sample), an interim analysis is performed on voxelotor whole blood and plasma concentration data for subjects in these 2 groups comparing data to historical data in healthy subjects to determine if there is an increase in exposure and if a dose adjustment is therefore needed for subjects with severe hepatic impairment. The subjects with normal hepatic function are enrolled last, once at least 4 subjects with severe hepatic impairment are enrolled.

Subjects remain in the clinical research unit (CRU) until discharge on Day 5 and return to the CRU on Days 12 (±1 day) and 20 (±1 day) for voxelotor whole blood and plasma PK and safety assessments. All subjects return for a follow-up visit on Day 28 (±2 days).

Eligible subjects receive a single oral dose of voxelotor on Day 1 (for normal hepatic function, mild and moderate hepatic impairment). A lower dose of voxelotor may be administered for the subjects with severe hepatic impairment.

Adverse events and concomitant medication use are monitored throughout the study. Safety assessments, including physical examinations, vital signs assessments, 12-lead ECGs, and clinical laboratory tests, are performed. Whole blood and plasma concentrations of voxelotor are determined using validated assays.

Pharmacokinetic variables are calculated from the whole blood and plasma concentrations of voxelotor using noncompartmental methods (Phoenix WinNonlin®, Version 6.3.0395, Pharsight Corp, St. Louis, MO) and actual sampling times. The following PK parameters are determined (Table 5).

TABLE 5

| | |
|---|---|
| AUC0-96 | Area under the concentration-time curve from time 0 to 96 hours; calculated using |
| | the linear/log trapezoid rule |
| AUCinf | Area under the concentration-time curve from time 0 extrapolated to infinity; |
| | calculated as AUCt + Ct/λz, where Ct was the last quantifiable concentration and |
| | λz was the terminal elimination rate constant determined by the slope of the |
| | terminal phase of the concentration-time curve |
| AUCinf, u | Unbound AUCinf for plasma |
| AUCt | Area under the concentration-time curve from time 0 to the time of the last |
| | quantifiable concentration; calculated using the linear/log trapezoid rule |
| AUCt, u | Unbound AUCt for plasma |
| CL/F | Apparent oral clearance |
| CL/F, u | Unbound CL/F for plasma |
| Cmax | Maximum observed concentration |
| Cmax, u | Unbound Cmax for plasma |
| Fu | Fraction of drug unbound in plasma |
| λz | Terminal elimination rate constant |
| t½ | Terminal elimination half-life; calculated as ln(2)/λz |
| tmax | The time that Cmax was observed |
| Vz/F | Apparent volume of distribution during the terminal phase |

### Example 3

The effect of voxelotor on the pharmacokinetics (PK) of a single dose of caffeine, warfarin sodium, omeprazole, and midazolam hydrochloride, which are probe substrates for cytochrome P450 (CYP)1A2, CYP2C9, CYP2C19, and CYP3A4, respectively, were studied in healthy subjects. During Period 1, subjects received a single dose of caffeine 100 mg, warfarin sodium 10 mg+vitamin K 10 mg, omeprazole 20 mg, and midazolam hydrochloride 2 mg on Day 1. During Period 2, subjects received voxelotor 900 mg QD on Days 1 and 2 followed by 600 mg QD on Day 3, a single dose of caffeine 100 mg, warfarin sodium 10 mg+vitamin K 10 mg, omeprazole 20 mg, and midazolam hydrochloride 2 mg on Day 4, and voxelotor 600 mg QD on Days 4 through 7.

Whole blood and plasma concentrations of voxelotor and plasma concentrations of caffeine, paraxanthine, S-warfarin, omeprazole, 5-hydroxyomeprazole, midazolam, and 1-hydroxymidazolam were determined using validated assays. Pharmacokinetic variables were calculated from the whole blood and plasma concentrations using noncompartmental methods including: maximum observed plasma concentration (Cₘₐₓ), area under the plasma concentration-time curve (AUC) from time 0 to the time of the last quantifiable concentration (AUCₜ), AUC from time 0 extrapolated to infinity (AUC_{inf}), the time that Cₘₐₓ was observed (tₘₐₓ), and terminal elimination half-life (t_{1/2}) for caffeine, paraxanthine, S-warfarin, omeprazole, 5-hydroxyomeprazole, midazolam, and 1-hydroxymidazolam; ratio of metabolite to parent Cₘₐₓ, AUCₜ, and AUC_{inf} corrected for molecular weight for paraxanthine/caffeine, 5-hydroxyomeprazole/omeprazole, and 1-hydroxymidazolam/midazolam in plasma; and Cₘₐₓ, tₘₐₓ, AUC from time 0 to 24 hours (Days 4 and 7), and t_{1/2} (Day 7) for voxelotor in whole blood and plasma.

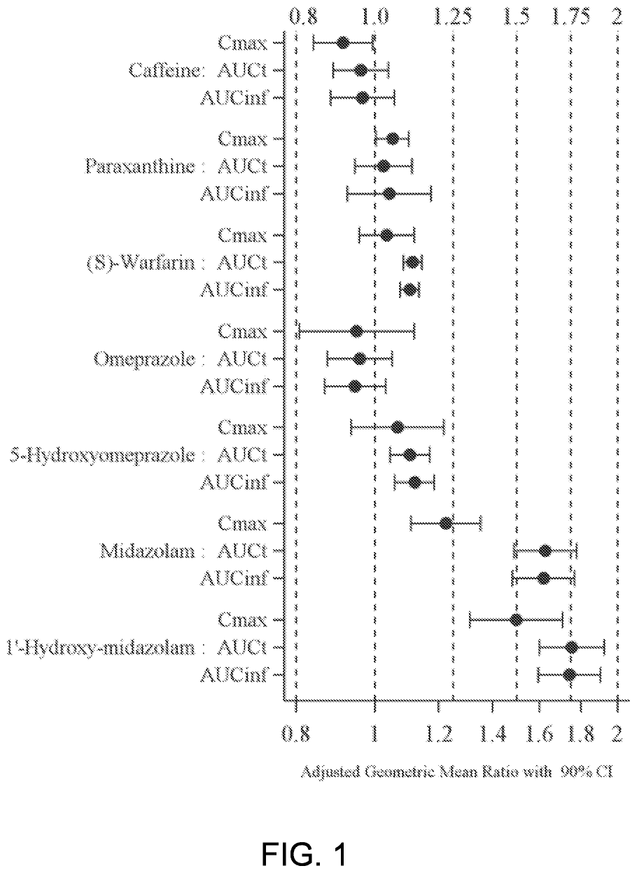

The effect of multiple doses on voxelotor on the PK of probe substrates for CYP1A2 (caffeine), CYP2C9 (S-warfarin), CYP2C19 (omeprazole), and CYP3A4 (midazolam) and metabolites paraxanthine, 5-hydroxyomeprazole, and 1-hydroxymidazolam is presented in FIG. 1.

From these studies, it was determined that the administration of voxelotor had no clinically meaningful effect on the PK of probe substrates for CYP1A2 (caffeine), CYP2C9 (S-warfarin), or CYP2C19 (omeprazole). Overall exposure of the CYP3A4 probe substrate (midazolam) was increased by 63% in the presence of voxelotor. The overall exposure of the midazolam metabolite, 1′-hydroxy-midazolam, was increased by 75% in the presence of voxelotor. Whole blood voxelotor exposure was approximately 20 to 25 times higher than plasma voxelotor exposure. Voxelotor was safe and well tolerated when administered alone and in combination with caffeine, warfarin sodium, omeprazole, and midazolam hydrochloride to healthy subjects.

A CYP3A4-mediated time dependent inhibition PBPK model (described in Example 3) was used to simulate the effects of voxelotor on the kinetics of midazolam. Specifically, simulated plasma concentrations of midazolam in the absence and presence of voxelotor (1500 mg QD) in patients with sickle cell disease were performed. The results are shown in Table 6.

Based on this data, it is contemplated that voxelotor is a moderate inhibitor of CYP3A4.

TABLE 6
Simulated Geometric Mean Ratios for Cₘₐₓ and AUC of midazolam in the
absence and presence of voxelotor (1500 mg QD) in patients with SCD

| | Control | | Presence of Voxelotor | | | |
|---|---|---|---|---|---|---|
| | Cₘₐₓ | AUC | Cₘₐₓ | AUC | Ratio | |
| | (ng/mL) | (ng/mL*h) | (ng/mL) | (ng/mL*h) | Cₘₐₓ | AUC |
| Simulated | 7.04 | 22.23 | 14.44 | 69.67 | 2.05 | 3.13 |
| | | | | | (2.02-2.17) | (2.95-3.47) |

### Example 4

PBPK modeling and simulation was also used to evaluate voxelotor as an object (victim) of drug interactions.

The relative contributions of enzymes to the formation of oxidative, glucuronidation, and reduction products of voxelotor were assigned on the basis of in vitro metabolism data and in vivo mass balance/metabolite profile data from previous studies, and a base model for PK in healthy subjects was developed using previous single-dose PK studies. Model verification was performed using clinical datasets from previous multiple-dose DDI studies in healthy subjects. Such previous studies were carried out according to methods known in the art. Subsequently, the model was refined and adjusted to describe the PK of voxelotor in subjects with sickle cell disease. Literature data on biochemical and physiological parameters likely to be affected by the disease and clinical data were used.

A PBPK model incorporated in a Simcyp simulator that considers both liver and intestinal metabolism was used, as it described the disposition of voxelotor with reasonable accuracy when compared with clinical data. The modeling studies were performed using a dose of 1500 mg of voxelotor.

Results for modeling studies of voxelotor, administered in a single dose, in the absence and presence of CYP3A4 modulators are summarized in Table 7. Results for modeling studies of voxelotor, administered in multiple doses, in the absence and presence of CYP3A4 modulators are summarized in Table 8. In these tables, “fmCYP3A4” refers to fraction metabolized by CYP3A4.

TABLE 7
Predicted Geometric Mean (GM) Ratios* for Plasma and Whole Blood
Cₘₐₓ and AUC of Voxelotor (Single Dose) in the Absence and Presence
of CYP3A4 Modulators (Multiple Dose) in SCD patients

Ratio

| CYP3A4 Modulator | GM Cₘₐₓ | GM AUC |
|---|---|---|
| Ketoconazole (400 mg) | 1.05 | 1.83 |
| Strong CYP3A4 inhibitor | (1.04-1.05) | (1.75-1.91) |
| Fluconazole (400 mg) | 1.05 | 2.16 |
| Moderate CYP3A4 inhibitor | (1.04-1.05) | (2.09-2.24) |
| Fluconazole (200 mg) | 1.04 | 1.84 |
| Moderate CYP3A4 inhibitor | (1.04-1.04) | (1.79-1.89) |
| Fluvoxamine (50 mg) | 1.02 | 1.20 |
| Weak CYP3A4 inhibitor | (1.01-1.02) | (1.19-1.22) |
| Rifampicin (600 mg) | 0.86 | 0.28 |
| Strong CYP3A4 inducer | (0.84-0.87) | (0.26-0.30) |
| Efavirenz (600 mg) | 0.94 | 0.46 |
| Moderate CYP3A4 inducer | (0.93-0.94) | (0.44-0.50) |

*Geometric mean ratios for whole blood are identical; assumes fmCYP3A4 0.75.

TABLE 8
Predicted Geometric Mean (GM) Ratios* for Plasma
and Whole Blood Cₘₐₓ and AUC of Voxelotor (Multiple
Doses) in the Absence and Presence of CYP3A4 Modulators
(Multiple Dose) in SCD patients

| | Ratio | | |
|---|---|---|---|
| CYP3A4 Modulator | GM Cₘₐₓ | GM Cₘᵢₙ | GM AUC |
| Ketoconazole (400 mg) | 1.40 | 1.52 | 1.46 |
| Strong CYP3A4 inhibitor | (1.35-1.45) | (1.46-1.59) | (1.46-1.59) |
| Fluconazole (400 mg) | 1.70 | 1.94 | 1.80 |
| Moderate CYP3A4 inhibitor | (1.64-1.77) | (1.85-2.04) | (1.73-1.88) |
| Fluconazole (200 mg) | 1.55 | 1.74 | 1.63 |
| Moderate CYP3A4 inhibitor | (1.51-1.60) | (1.67-1.82) | (1.58-1.69) |
| Fluvoxamine (50 mg) | 1.22 | 1.28 | 1.24 |
| Weak CYP3A4 inhibitor | (1.19-1.24) | (1.25-1.31) | (1.22-1.27) |
| Rifampicin (600 mg) | 0.33 | 0.11 | 0.23 |
| Strong CYP3A4 inducer | (0.31-0.35) | (0.09-0.14) | (0.21-0.25) |
| Efavirenz (600 mg) | 0.48 | 0.31 | 0.40 |
| Moderate CYP3A4 inducer | (0.44-0.51) | (0.28-0.35) | (0.37-0.44) |

*Geometric mean ratios for whole blood are identical; assumes fmCYP3A4 0.75.

Based on the above data, it is contemplated that co-administration of moderate or strong CYP3A4 inducers can decrease voxelotor plasma and whole blood exposures (Cₘᵢₙ, Cₘₐₓ, and AUC), which can thereby reduce the efficacy of voxelotor. It is further contemplated that co-administration of strong CYP3A4 inhibitors can increase plasma and whole blood exposures (Cₘₐₓ and AUC).

### Example 5

### 1 Indications and Usage

OXBRYTA is indicated for the treatment of sickle cell disease (SCD) in adults and pediatric patients 4 years of age and older.

This indication is approved under accelerated approval based on increase in hemoglobin (Hb) [see Clinical Studies (14)]. Continued approval for this indication may be contingent upon verification and description of clinical benefit in confirmatory trial(s).

### 2 Dosage and Administration

### 2.1 Recommended Dosage for Adults and Pediatric Patients 12 Years and Older

The recommended dosage of OXBRYTA is 1,500 mg orally once daily.

### 2.2 Recommended Dosage for Pediatric Patients 4 Years to Less Than 12 Years

For pediatric patients 4 years to less than 12 years, select the appropriate product (OXBRYTA tablets or OXBRYTA tablets for oral suspension) based on patient's ability to swallow tablets and patient's weight.

The recommended dosage of OXBRYTA for pediatric patients 4 years to less than 12 years is shown in Table 9.

TABLE 9
Recommended OXBRYTA Dosage in Pediatric
Patients 4 Years to Less Than 12 Years

| | |
|---|---|
| Body Weight | Recommended Dose (once daily) |

| | | |
|---|---|---|
| 40 kg or greater | 1,500 | mg |
| 20 kg to less than 40 kg | 900 | mg |
| 10 kg to less than 20 kg | 600 | mg |

2.3 Recommended Dosage for Adults and Pediatric Patients 12 Years and Older with Hepatic Impairment

The recommended dosage of OXBRYTA in adults and pediatric patients 12 years and older with severe hepatic impairment (Child Pugh C) is 1,000 mg orally once daily.

No dosage adjustment of OXBRYTA is required for patients with mild or moderate hepatic impairment [see Use in Specific Populations (8.6) and Clinical Pharmacology (12.3)].

2.4 Recommended Dosage for Pediatric Patients 4 Years to Less than 12 Years with Hepatic Impairment

The recommended dosage of OXBRYTA in pediatric patients 4 years to less than 12 years with severe hepatic impairment (Child Pugh C) is described in Table 10.

No dosage adjustment of OXBRYTA is required for patients with mild or moderate hepatic impairment [see Use in Specific Populations (8.6) and Clinical Pharmacology (12.3)].

TABLE 10
Recommended OXBRYTA Dosage in Pediatric Patients 4 Years to Less
Than 12 Years with Severe Hepatic Impairment (Child Pugh C)

| Body Weight | Recommended Dose (once daily) |
|---|---|
| 40 kg or greater | 1,000 mg (two 500 mg tablets) or |
| | 900 mg (three 300 mg tablets for oral |
| | suspension or three 300 mg tablets) |
| 20 kg to less than 40 kg | 600 mg |
| 10 kg to less than 20 kg | 300 mg |

2.5 Recommended Dosage of OXBRYTA for Adults and Pediatric Patients 12 Years and Older when Used with Concomitant Strong or Moderate CYP3A4 Inducers

### CYP3A4 Inducers

Avoid concomitant use of strong or moderate CYP3A4 inducers with OXBRYTA [see Drug Interactions (7.1) and Clinical Pharmacology (12.3)].

If concomitant use of strong CYP3A4 inducers is unavoidable, the recommended dosage of OXBRYTA is 2,500 mg orally once daily. If concomitant use of moderate CYP3A4 inducers is unavoidable, the recommended dosage of OXBRYTA is 2,000 mg orally once daily.

2.6 Recommended Dosage of OXBRYTA for Pediatric Patients 4 Years to Less than 12 Years when Used with Concomitant Strong or Moderate CYP3A4 Inducers

### CYP3A4 Inducers

Avoid concomitant use of strong or moderate CYP3A4 inducers with OXBRYTA [see Drug Interactions (7.1) and Clinical Pharmacology (12.3)]. If concomitant use of strong or moderate CYP3A4 inducers is unavoidable, see Table 11 for dosage.

TABLE 11
OXBRYTA Recommended Dosage for Pediatric Patients 4 Years to Less Than
12 Years When Used with Concomitant Strong or Moderate CYP3A4 Inducers

| | Recommended Dose (once daily) | | | |
|---|---|---|---|---|
| | Concomitant Use of Strong | | Concomitant Use of Moderate | |
| Body Weight | CYP3A4 Inducers | | CYP3A4 Inducers | |
| 40 kg or greater | 2,500 mg (five 500 mg tablets) or | | 2,000 mg (four 500 mg tablets) or | |
| | 2,400 mg (eight 300 mg tablets | | 2,100 mg (seven 300 mg tablets | |
| | for oral suspension or eight 300 | | for oral suspension or seven 300 | |
| | mg tablets) | | mg tablets) | |
| 20 kg to less than 40 kg | 1,500 | mg | 1,200 | mg |
| 10 kg to less than 20 kg | 900 | mg | 900 | mg |

### 2.7 Important Administration Instructions

Administer OXBRYTA orally, once daily with or without food. If a dose is missed, or not administered entirely, resume dosing the following day.

OXBRYTA may be given with or without hydroxyurea.

### OXBRYTA 300 mg and 500 mg Tablets

Patients should swallow OXBRYTA tablets whole. Do not cut, crush, or chew the tablets.

### OXBRYTA 300 mg Tablets for Oral Suspension

Patients should disperse tablets for oral suspension immediately before administration in a cup and in room temperature clear liquid (such as drinking water, clear soda, apple juice, clear electrolyte drinks, clear flavored drinks, or clear sports drinks) before swallowing.

Do not swallow whole, cut, crush, or chew the tablets for oral suspension.

TABLE 12

| Recommended | | Number of Tablets for | Minimum Recommended | |
|---|---|---|---|---|
| Daily Dose | | Oral Suspension | Volume of Clear Drink | |
| 300 | mg | 1 | 5 mL | (1 teaspoon) |
| 600 | mg | 2 | 10 mL | (2 teaspoons) |
| 900 | mg | 3 | 15 mL | (3 teaspoons) |
| 1,200 | mg | 4 | 20 mL | (4 teaspoons) |
| 1,500 | mg | 5 | 25 mL | (5 teaspoons) |
| 2,100 | mg | 7 | 35 mL | (7 teaspoons) |
| 2,400 | mg | 8 | 40 mL | (8 teaspoons) |

After the tablets start to disintegrate, swirl the contents of the cup until the tablets are dispersed, wait 1 to 5 minutes, swirl the contents of the cup again, and then orally administer the contents of the cup. The tablet(s) will not completely dissolve; there will still be small tablet clumps in the mixture.

Resuspend any residue left in the cup in more clear drink and administer. Repeat until no tablet residue is left in the cup.

Tablets for oral suspension may be substituted for tablets in adults and pediatric patients 12 years and older with difficulty swallowing the tablets. Use the number of tablets for oral suspension needed to achieve the recommended dose.

### 3 Dosage Forms and Strengths

Tablets: 300 mg light purple to purple, oval shaped, biconvex, debossed with “G 300” on one side.

Tablets: 500 mg light yellow to yellow, oval shaped, biconvex, debossed with “GBT 500” on one side.

Tablets for oral suspension: 300 mg light yellow to yellow, round shaped, debossed with “300 D” on one side.

### 4 Contraindications

OXBRYTA is contraindicated in patients with a history of serious drug hypersensitivity reaction to voxelotor or excipients. Clinical manifestations may include generalized rash, urticaria, mild shortness of breath, mild facial swelling, and eosinophilia [see Warnings and Precautions (5.1) and Adverse Reactions (6.1)].

### 5 Warnings and Precautions

### 5.1 Hypersensitivity Reactions

Serious hypersensitivity reactions after administration of OXBRYTA have occurred in <1% of patients treated. Clinical manifestations may include generalized rash, urticaria, mild shortness of breath, mild facial swelling, and eosinophilia [see Adverse Reactions (6.1)].

If hypersensitivity reactions occur, discontinue OXBRYTA and administer appropriate medical therapy. Do not reinitiate OXBRYTA in patients who experience these symptoms with previous use.

### 5.2 Laboratory Test Interference

OXBRYTA administration may interfere with measurement of Hb subtypes (HbA, HbS, and HbF) by high-performance liquid chromatography (HPLC) [see Drug Interactions (7.3)]. If precise quantitation of Hb species is required, chromatography should be performed when the patient has not received OXBRYTA therapy in the immediately preceding 10 days.

### 6 Adverse Reactions

The following clinically significant adverse reaction is discussed in other sections of the labeling: Hypersensitivity Reactions [see Contraindications (4) and Warnings and Precautions (5.1)].

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Adults and Pediatric Patients 12 Years and Older

The safety of OXBRYTA was evaluated in the HOPE trial based on data from 88 patients with SCD who received OXBRYTA 1,500 mg and 91 patients who received placebo orally once daily [see Clinical Studies (14.1)]. Seventy-four patients received OXBRYTA 1,500 mg once daily for ≥24 weeks, 65 patients for ≥48 weeks, and 63 patients completed the 72-week treatment period.

In patients who received OXBRYTA 1,500 mg once daily, the median age was 24 years (range:12 to 59 years); 65% female; 66% Black or African American and 23% Arab/Middle Eastern; and 65% receiving hydroxyurea at baseline.

Serious adverse reactions occurred in 3% ( 3/88) of patients receiving OXBRYTA 1,500 mg, which included headache, drug hypersensitivity, and pulmonary embolism occurring in 1 patient each. Permanent discontinuation due to an adverse reaction (Grades 1-4) occurred in 5% ( 4/88) of patients who received OXBRYTA 1,500 mg.

Dosage modifications (dose reduction or dosing interruption) due to an adverse reaction occurred in 48% ( 42/88) of patients who received OXBRYTA. Most frequent adverse reactions requiring dosage modifications occurring in more than two patients who received OXBRYTA 1,500 mg included diarrhea and rash.

The safety profile observed in pediatric patients 12 to <17 years treated with OXBRYTA in the HOPE trial was similar to that seen in adult patients.

The most common adverse reactions occurring in ≥10% of patients treated with OXBRYTA 1,500 mg with a difference of >3% compared to placebo are summarized in Table 13.

TABLE 13
Adverse Reactions (≥10%) in Patients Receiving OXBRYTA with a
Difference Between Arms of >3% Compared to Placebo in HOPE

| Adverse Reaction^{a} | OXBRYTA 1,500 mg (N = 88) | Placebo (N = 91) |
|---|---|---|
| Headache | 32% | 25% |
| Diarrhea | 23% | 11% |
| Abdominal Pain^{b} | 23% | 16% |
| Nausea | 19% | 10% |
| Rash^{c} | 15% | 11% |
| Pyrexia | 15% | 8% |

^{a}Adverse reactions were Grades 1 or 2 except for Grade 3 headache (2), diarrhea (1), nausea (1), rash (1), and rash generalized (3)
^{b}Abdominal pain (grouped PTs) includes the following PTs: abdominal pain, lower abdominal pain, and upper abdominal pain
^{c}Rash (grouped PTs) includes the following PTs: rash, urticaria, generalized rash, macular rash, maculopapular rash, pruritic rash, and papular rash

Clinically relevant adverse reactions occurring in <10% of patients included:

Drug hypersensitivity

### Pediatric Patients 4 to <12 Years

The safety of OXBRYTA in pediatric patients 4 to <12 years with SCD was evaluated in an open-label, Phase 2 study [see Clinical Studies (14.2)]. In this study, 45 patients 4 to <12 years of age received doses of OXBRYTA tablets for oral suspension based on weight at baseline. Thirty-five patients received OXBRYTA for 24 weeks and 26 patients for 48 weeks. The most common adverse reactions (>10%) reported in pediatric patients 4 to <12 years were pyrexia (36%), vomiting (33%), rash (20%), abdominal pain (18%), diarrhea (18%), and headache (18%).

The overall safety profile of OXBRYTA in pediatric patients 4 to <12 years was similar to that seen in adults and pediatric patients 12 years and older.

### 7 Drug Interactions

### 7.1 Effect of Other Drugs on Voxelotor

### Strong or Moderate CYP3A4 Inducers

Coadministration of strong or moderate CYP3A4 inducers may decrease voxelotor plasma and whole blood concentrations and may lead to reduced efficacy.

Avoid coadministration of OXBRYTA with strong or moderate CYP3A4 inducers. Increase the OXBRYTA dosage when coadministration with a strong or moderate CYP3A4 inducer is unavoidable [see Dosage and Administration (2.5, 2.6) and Clinical Pharmacology (12.3)].

### 7.2 Effect of Voxelotor on Other Drugs

Voxelotor increased the systemic exposure of midazolam (a sensitive CYP3A4 substrate) [see Clinical Pharmacology (12.3)]. Avoid coadministration of OXBRYTA with sensitive CYP3A4 substrates with a narrow therapeutic index. If concomitant use is unavoidable, consider dose reduction of the sensitive CYP3A4 substrate(s).

### 7.3 Laboratory Test Interference

OXBRYTA administration may interfere with measurement of Hb subtypes (HbA, HbS, and HbF) by HPLC [see Warnings and Precautions (5.2)]. If precise quantitation of Hb species is required, chromatography should be performed when the patient has not received OXBRYTA therapy in the immediately preceding 10 days.

### 8 Use in Specific Populations

### 8.1 Pregnancy

### Risk Summary

There are no available data on OXBRYTA use in pregnant women to evaluate for a drug-associated risk of major birth defects, miscarriage, or adverse maternal or fetal outcomes. In animal reproduction studies, oral administration of voxelotor to pregnant rats and rabbits during organogenesis at exposures up to 2.8-times (rats) and 0.3-times (rabbits) the exposure at the maximum recommended human dose resulted in no adverse developmental effects (see Data).

The estimated background risk of major birth defects and miscarriage for the indicated population is approximately 14% and up to 43%, respectively. All pregnancies have a background risk of birth defect, loss, or other adverse outcomes.

There are adverse effects on maternal and fetal outcomes associated with SCD in pregnancy (see Clinical Considerations). OXBRYTA should only be used during pregnancy if the benefit of the drug outweighs the potential risk.

### Clinical Considerations

Disease-Associated Maternal and/or Embryo/Fetal Risk

Women with SCD have an increased risk of adverse pregnancy outcomes for the mother and the fetus. Pregnant women are at greater risk for vaso-occlusive crises, pre-eclampsia, eclampsia, and maternal mortality. For the fetus, there is an increased risk for intrauterine growth restriction, preterm delivery, low birth weight, and perinatal mortality.

### Data

### Animal Data

In embryo-fetal development studies, voxelotor was administered orally to pregnant rats at 15, 50, and 250 mg/kg/day (gestation days 7 through 17) and rabbits at 25, 75, and 150 mg/kg/day (gestation days 7 through 19) through organogenesis. Maternal toxicity was observed at the highest dose levels in these studies equivalent to 2.8-times (rats) and 0.3-times (rabbits) the exposures in patients receiving OXBRYTA at the recommended daily dose. There was no evidence of adverse developmental outcomes in rats or rabbits

In a pre- and postnatal development study, voxelotor was administered orally to pregnant rats at 15, 50 and 250 mg/kg/day (gestation day 6 through lactation day 20). Maternal gestational body weights were decreased at 250 mg/kg/day, which continued to the end of lactation. The findings in offspring included reduced survival and reduced body weights throughout lactation, weaning and maturation. The effects in offspring were observed at the maternal dose of 250 mg/kg/day with an exposure approximately 2.8-times the exposure in patients at the recommended dose.

### 8.2 Lactation

### Risk Summary

There are no data on the presence of voxelotor in human milk, the effects on the breastfed child, or the effects on milk production. Voxelotor was detected in milk in lactating rats. Plasma concentrations of voxelotor in pregnant rats were higher than the concentration in milk. When a drug is present in animal milk, it is likely that the drug will be present in human milk. The concentration of voxelotor in animal milk does not necessarily predict the concentration of drug in human milk. Because of the potential for serious adverse reactions in the breastfed child, including changes in the hematopoietic system, advise patients that breastfeeding is not recommended during treatment with OXBRYTA, and for at least 2 weeks after the last dose.

### 8.4 Pediatric Use

The safety and effectiveness of OXBRYTA for SCD have been established in pediatric patients aged 4 years and older. The safety and efficacy of OXBRYTA in pediatric patients with SCD below the age of 4 years have not been established.

Use of OXBRYTA in pediatric patients 12 to <17 years for SCD is supported by evidence from an adequate and well-controlled study in adults and pediatric patients (HOPE trial). The HOPE trial enrolled 26 pediatric patients aged 12 to <17 years, in which 12 pediatric patients received OXBRYTA 1,500 mg once daily and 14 pediatric patients received OXBRYTA 900 mg once daily [see Adverse Reactions (6.1), Clinical Pharmacology (12.3), and Clinical Studies (14.1)].

Use of OXBRYTA in pediatric patients 4 to <12 years for SCD is supported by evidence from an open-label, Phase 2 study. The study enrolled 45 pediatric patients aged 4 to <12 years and 11 patients aged 12 to <17 years with SCD. Patients 12 to <17 years received OXBRYTA 1,500 mg once daily. Patients 4 to <12 years were administered OXBRYTA based on body weight. OXBRYTA doses of 600 mg, 900 mg, or 1,500 mg once daily were administered to patients weighing 10 kg to <20 kg, 20 kg to <40 kg, or ≥40 kg, respectively [see Adverse Reactions (6.1), Clinical Pharmacology (12.3), and Clinical Studies (14.2)].

Pharmacokinetics, safety and efficacy were similar across the pediatric age groups and across pediatric and adult patients [see Dosage and Administration (2), Clinical Pharmacology (12.3) and Clinical Studies (14)].

The adverse reactions observed were similar across the pediatric age groups and across pediatric and adult patients [see Adverse Reactions (6.1)].

### 8.5 Geriatric Use

Clinical studies of OXBRYTA did not include sufficient numbers of subjects aged 65 and over to determine whether they respond differently from younger subjects.

### 8.6 Hepatic Impairment

Severe hepatic impairment increases voxelotor exposures [see Clinical Pharmacology (12.3)]. Reduce OXBRYTA dose [see Dosage and Administration (2.3, 2.4)].

### 11 Description

OXBRYTA contains voxelotor, a hemoglobin S polymerization inhibitor. The chemical name of voxelotor is 2-hydroxy-6-((2-(1-isopropyl-1H-pyrazol-5-yl)pyridin-3-yl)methoxy)benzaldehyde with a molecular formula of C19H19N3O3 and a molecular weight of 337.4.

The chemical structure of voxelotor is:

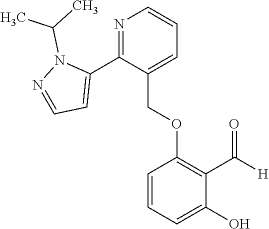 [SMILES: CC(C)n1nccc1-c1ncccc1COc1cccc(O)c1C=O]

Voxelotor is a white-to-yellow-to-beige compound in crystalline Form II of its free base. It is non-hygroscopic and highly soluble in common organic solvents such as acetone and toluene and insoluble in water.

OXBRYTA film-coated tablets for oral use contain either 300 mg or 500 mg of voxelotor. Both strengths of OXBRYTA film-coated tablets contain the following inactive ingredients: colloidal silicon dioxide, croscarmellose sodium, magnesium stearate, microcrystalline cellulose, and sodium lauryl sulfate. In addition, the 500 mg tablet film coating contains: polyethylene glycol 3350, polyvinyl alcohol, talc, titanium dioxide, and yellow iron oxide. The 300 mg tablet film coating contains: black and red iron oxide, polyethylene glycol 3350, polyvinyl alcohol, talc, and titanium dioxide.

Each OXBRYTA tablet for oral suspension contains 300 mg of voxelotor with the following inactive ingredients: artificial grape flavor, colloidal silicon dioxide, croscarmellose sodium, iron oxide pigment, magnesium stearate, microcrystalline cellulose, and sucralose.

### 12 Clinical Pharmacology

### 12.1 Mechanism of Action

Voxelotor is a hemoglobin S (HbS) polymerization inhibitor that binds to HbS with a 1:1 stoichiometry and exhibits preferential partitioning to red blood cells (RBCs). By increasing the affinity of Hb for oxygen, voxelotor demonstrates dose-dependent inhibition of HbS polymerization. Nonclinical studies suggest that voxelotor may inhibit RBC sickling, improve RBC deformability, and reduce whole blood viscosity.

### 12.2 Pharmacodynamics

The pharmacodynamic effect of voxelotor treatment demonstrated a dose-dependent increase in Hb oxygen affinity as determined by the change in p50 (partial pressure of oxygen at which Hb oxygen saturation of 50% is achieved) that was linearly correlated with voxelotor exposure.

The pharmacodynamic effect of voxelotor treatment also demonstrated a dose-dependent reduction in clinical measures of hemolysis (indirect bilirubin and % reticulocytes).

### Cardiac Electrophysiology

At plasma concentrations approximately 2-fold above therapeutic concentrations, voxelotor does not prolong QT interval to any clinically relevant extent.

### 12.3 Pharmacokinetics

Voxelotor is absorbed into plasma and is then distributed predominantly into RBCs due to its preferential binding to Hb. The major route of elimination of voxelotor is by metabolism with subsequent excretion of metabolites into urine and feces. The PK are linear and voxelotor exposures increased proportionally with either single or multiple doses (Table 14) in whole blood, plasma, and RBCs. Steady-state after repeated administration is reached within 8 days and exposures of voxelotor are consistent with accumulation predicted based on single dose data in patients with SCD.

TABLE 14
Pharmcokinetics Parameters of Voxelotor
in Plasma and Whole Blood^{a}

| | |
|---|---|
| | Voxelotor 1,500 mg |
| PK Parameter | Geometric Mean (% CV) |

Plasma PK

| | | |
|---|---|---|
| AUC0-24 h (μg · hr/mL) | 278 | (28.4) |
| Cmax (μg/mL) | 14 | (24.5) |
| Half-life (hours) | 38.7 | (30.2) |

Whole Blood PK

| | | |
|---|---|---|
| AUC0-24 h (μg · hr/mL) | 3,830 | (33.5) |
| Cmax (μg/mL) | 180 | (31) |

^{a}Based on the 72-week population pharmacokinetic analysis.

In healthy subjects, voxelotor exposures were comparable when administered as tablet for oral suspension dispersed in water or as oral tablet swallowed whole.

### Absorption

The median plasma and whole blood Tₘₐₓ of voxelotor after oral administration is 2 hours. The mean peak concentrations in whole blood and RBCs are observed between 6 and 18 hours after oral administration.

### Effect of Food

A high-fat, high-calorie meal increased voxelotor AUC by 42% and Cmax by 45% in whole blood relative to AUC and Cmax in the fasted state. Similarly, AUC increased by 42% and Cmax increased by 95% in plasma.

### Distribution

Voxelotor apparent volume of distribution of the central compartment and peripheral compartment are 333 L and 72.3 L in plasma, respectively. Protein binding is 99.8% in vitro. The blood-to-plasma ratio is approximately 17:1 in patients with SCD.

### Elimination

The geometric mean (% CV) terminal elimination half-life of voxelotor in patients with SCD is 38.7 hours (30.2%) with concentrations in plasma, whole blood, and RBCs declining in parallel. The apparent oral clearance of voxelotor was estimated as 6.1 L/h in plasma in patients with SCD.

### Metabolism

In vitro and in vivo studies indicate that voxelotor is extensively metabolized through Phase I (oxidation and reduction), Phase II (glucuronidation) and combinations of Phase I and II metabolism. Metabolism of voxelotor is mediated by CYP3A4, CYP3A5, CYP2B6, CYP2C19, CYP2C9, UGT1A1, and UGT1A9.

### Excretion

Following the administration of radiolabeled voxelotor, approximately 62.6% of the dose and its metabolites are excreted into feces (33.3% unchanged) and 35.5% in urine (0.08% unchanged).

### Specific Populations

No clinically significant differences in the pharmacokinetics of voxelotor were observed based on age (12 to 59 years), sex, body weight (28 to 135 kg), or mild to severe renal impairment (creatinine clearance [CLcr]15-89 mL/min).

### Pediatric Patients

The pharmacokinetic exposures of voxelotor in whole blood and plasma were similar between pediatric patients 4 to <17 years and adults following the recommended dosage [see Dosage and Administration (2)].

Patients with Renal Impairment

There was no clinically significant effect of renal function on the excretion of voxelotor. Following a single 900 mg dose of voxelotor, whole blood exposures in subjects with severe renal impairment (eGFR <30 mL/min/1.73 m²) were 25% lower compared to healthy controls.

The unbound plasma concentrations were comparable. OXBRYTA has not been evaluated in patients with end stage renal disease requiring dialysis.

Patients with Hepatic Impairment

The voxelotor AUC in whole blood were 14% and 15% higher in subjects with mild and moderate hepatic impairment (Child Pugh A and B) and 90% higher in subjects with severe hepatic impairment (Child Pugh C) compared to subjects with normal hepatic function.

Patients with HbSC Genotype

Voxelotor steady state whole blood AUC and Cₘₐₓ were 50% and 45% higher in HbSC genotype patients (n=11) compared to HbSS genotype (n=220) patients and voxelotor steady state plasma AUC and Cₘₐₓ were 23% and 15% higher in HbSC genotype patients compared to HbSS genotype patients.

### Drug Interaction Studies

### Clinical Studies and Model-Informed Approaches

Effect of Strong CYP3A4 Inhibitors on Voxelotor: concomitant use of OXBRYTA with itraconazole increased voxelotor AUC in healthy subjects by 11%.

Effect of Strong or Moderate CYP3A4 Inducers on Voxelotor: concomitant use of OXBRYTA with rifampin (a strong CYP3A4 inducer) is predicted to decrease voxelotor AUC in patients by up to 40%, and efavirenz (a moderate CYP3A4 inducer) is predicted to decrease voxelotor AUC in patients by up to 24%.

Effect of Acid Reducing Agents on Voxelotor, coadministration of omeprazole (proton pump inhibitor) with OXBRYTA did not alter voxelotor exposure.

Effect of Voxelotor on CYP450 Enzymes: in vivo voxelotor inhibits CYP3A4, but not CYP1A2, CYP2C9, CYP2C19, CYP2C8, or CYP2D6. The observed exposure increase of the CYP3A4 substrate midazolam in healthy subjects was 1.6-fold and the predicted increase in patients after multiple dosing is 2.5-fold.

Effect of Voxelotor on P-gp: concomitant use of OXBRYTA with digoxin (a P-gp substrate) did not alter digoxin to a clinically relevant extent.

### In Vitro Studies

CYP Enzymes: voxelotor is a reversible and time-dependent inhibitor as well as an inducer of CYP2B6.

Transporter Systems: voxelotor is not an inhibitor of P-gp, BCRP, OATP1B1, OATP1B3, OCT2, OAT1, OAT3, MATE1, MATE2-K, or BSEP. Voxelotor is not a substrate of P-gp, BCRP, OATP1A2, OATP1B1, OATP1B3, or BSEP.

### 13 Nonclinical Toxicology

### 13.1 Carcinogenesis, Mutagenesis, Impairment of Fertility

Voxelotor was not carcinogenic in a 26-week study in RasH2 transgenic mice at oral doses of 30, 150, or 500 mg/kg/day.

Voxelotor was not genotoxic in the reverse mutation bacterial (Ames) test, rat Comet assay, or rat micronucleus assay.

Voxelotor was not genotoxic in the reverse mutation bacterial (Ames) test, rat Comet assay, or rat micronucleus assay.

In a fertility and early embryonic development study, voxelotor was administered orally to rats at 15, 50, and 250 mg/kg/day. Males were dosed 28 days prior to mating through cohabitation and females were dosed 14 days prior to mating through gestation Day 7. Voxelotor had no effect on fertility or reproductive function. Sperm motility was decreased and changes in sperm morphology occurred at 250 mg/kg/day (approximately 5-times the human exposure at 1,500 mg/day).

### 14 Clinical Studies

### 14.1 Adults and Pediatric Patients 12 Years and Older

The efficacy and safety of OXBRYTA in SCD was evaluated in HOPE, a Phase 3 randomized, double-blind, placebo-controlled, multicenter trial [NCT 03036813]. In this study, 274 patients were randomized to daily oral administration of OXBRYTA 1,500 mg (N=90), OXBRYTA 900 mg (N=92), or placebo (N=92). Patients were included if they had from 1 to 10 vaso-occlusive crisis (VOC) events within 12 months prior to enrollment and baseline hemoglobin (Hb) ≥5.5 to ≤10.5 g/dL. Eligible patients on stable doses of hydroxyurea for at least 90 days were allowed to continue hydroxyurea therapy throughout the study. Randomization was stratified by patients already receiving hydroxyurea (yes, no), geographic region (North America, Europe, Other), and age (12 to <17 years, 18 to 65 years). The trial excluded patients who received red blood cell (RBC) transfusions within 60 days and erythropoietin within 28 days of enrollment, had renal insufficiency, uncontrolled liver disease, were pregnant, or lactating.

The majority of patients had HbSS or HbS/beta⁰-thalassemia genotype (90%) and were receiving background hydroxyurea therapy (65%). The median age was 24 years (range: 12 to 64 years); 46 (17%) patients were 12 to <17 years. Median baseline Hb was 8.5 g/dL (5.9 to 10.8 g/dL). One hundred and fifteen (42%) had 1 VOC event and 159 (58%) had 2 to 10 events within 12 months prior to enrollment. In the OXBRYTA 1,500 mg group, 63 (70%) patients completed the study through Week 72.

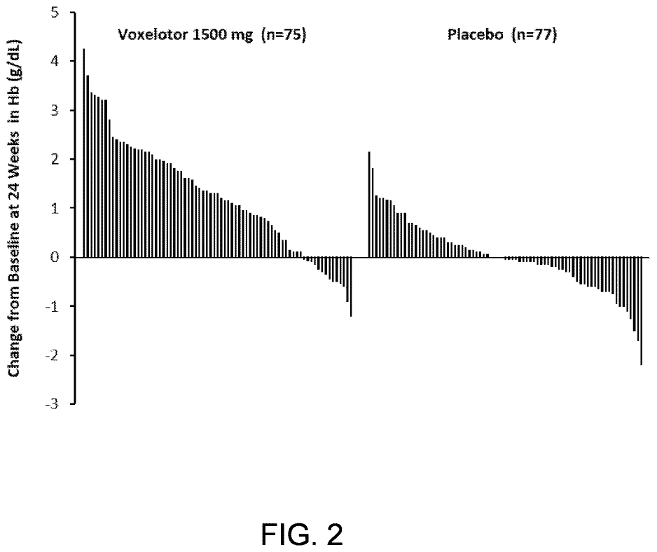

Efficacy was based on Hb response rate defined as a Hb increase of >1 g/dL from baseline to Week 24 in patients treated with OXBRYTA 1,500 mg versus placebo. The response rate for OXBRYTA 1,500 mg was 51.1% ( 46/90) compared to 6.5% ( 6/92) in the placebo group (p<0.001). No outlier subgroups were observed. The distribution of Hb change from baseline for individual patients completing 24 weeks of treatment with OXBRYTA 1,500 mg or placebo is depicted in FIG. 2.

Additional efficacy evaluation included change in Hb and percent change in indirect bilirubin and percent reticulocyte count from baseline to Week 24 (Table 15).

TABLE 15
Adjusted Mean (SE) Change from Baseline to Week 24
in Hemoglobin and Clinical Measures of Hemolysis

| | OXBRYTA 1,500 mg | | |
|---|---|---|---|
| | QD | Placebo | |
| | (N = 90) | (N = 92) | P Value |
| Hemoglobin | 1.1 g/dL | −0.1 g/dL | <0.001 |
| | (0.1) | (0.1) | |
| Indirect Bilirubin | −29.1% | −2.8% | <0.001 |
| | (3.5) | (3.5) | |
| Percent Reticulocyte | −18.0% | 6.8% | <0.001 |
| Count | (4.7) | (4.7) | |

QD = once daily;
SE = standard error

### 14.2 Pediatric Patients 4 to <12 Years

The efficacy and safety of OXBRYTA in patients 4 to <12 years with SCD was evaluated in an open-label, multi-center, Phase 2 trial [NCT 02850406]. In this study, 45 patients 4 to <12 years and 11 patients 12 to <17 years received OXBRYTA. Patients 4 to <12 years received tablets for oral suspension based on body weight at baseline. OXBRYTA doses of 600 mg, 900 mg, or 1,500 mg once daily were administered to patients weighing 10 kg to <20 kg, 20 kg to <40 kg, or ≥40 kg, respectively. Patients 12 to <17 years received OXBRYTA 1,500 mg once daily.

Patients were included if their baseline hemoglobin (Hb) was <10.5 g/dL. Eligible patients on stable doses of hydroxyurea for at least 90 days were allowed to continue hydroxyurea therapy throughout the study. The trial excluded patients who had a VOC event within 14 days prior to enrollment, received red blood cell (RBC) transfusions within 30 days of enrollment, and had renal insufficiency or uncontrolled liver disease.

All patients had HbSS or HbS/beta⁰-thalassemia genotype (100%) and most were receiving background hydroxyurea therapy (80%). The median age was 8 years (range: 4 to 15 years); 45 (80%) patients were 4 to <12 years. In this age group, mean baseline Hb was 8.6 g/dL (range: 6.1 to 10.5 g/dL).

Efficacy was based on Hb response rate, which is defined as a Hb increase of >1 g/dL from baseline to Week 24. Hb response rate for OXBRYTA in patients aged 4 to <12 years who took at least one dose of OXBRYTA was 36% ( 16/45) (95% CI: 21.6%, 49.5%).

### 16 How Supplied/Storage and Handling

The 300 mg tablet is film-coated, light purple to purple, oval shaped, biconvex, debossed with “G 300” on one side, available in:
Bottles of 60 tablets with one desiccant canister, a polyester coil and child-resistant closure: NDC 72786-102-02
Bottles of 90 tablets with one desiccant canister, a polyester coil and child-resistant closure: NDC 72786-102-03

The 500 mg tablet is film-coated, light yellow to yellow, oval shaped, biconvex, debossed with “GBT 500” on one side, and available in:
Bottles of 90 tablets with one desiccant canister, a polyester coil and child-resistant closure: NDC 72786-101-01

The 300 mg tablet for oral suspension is light yellow to yellow, round shaped, debossed with “300 D” on one side, and available in:
Bottles of 60 tablets for oral suspension with a polyester coil and child-resistant closure: NDC 72786-111-02
Bottles of 90 tablets for oral suspension with a polyester coil and child-resistant closure: NDC 72786-111-03

Do not eat the desiccant canister or the polyester coil.

Store OXBRYTA at 20° C. to 30° C. (68° F. to 86° F.).

### 17 Patient Counseling Information

Advise the patient to read the FDA-approved patient labeling (Patient Information and Instructions for Use).

### Hypersensitivity Reactions

Advise patients that serious hypersensitivity reactions may occur, and to notify their healthcare providers if they develop generalized rash, urticaria, shortness of breath, facial swelling and eosinophilia [see Warnings and Precautions (5.1)].

### Breastfeeding

Advise women not to breastfeed while they are on OXBRYTA therapy [see Use in Specific Populations (8.2)].

### Dosage and Administration

To avoid a dosing error from using the wrong formulation of OXBRYTA, strongly advise patients and caregivers to visually inspect the tablets to verify the correct formulation each time the prescription is filled [see Dosage and Administration (2) and How Supplied/Storage and Handling (16)].

Advise patients to:
Store OXBRYTA at 20° C. to 30° C. (68° F. to 86° F.).
Continue taking OXBRYTA every day for as long as their physician tells them.
Do not take St John's wort while taking OXBRYTA.
Swallow OXBRYTA tablets whole. Do not cut, crush, or chew the tablets.
Do not swallow whole, cut, crush, or chew OXBRYTA tablets for oral suspension. Disperse OXBRYTA tablets for oral suspension in room temperature clear drink (such as drinking water, clear soda, apple juice, clear electrolyte drinks, clear flavored drinks, or clear sports drinks) before administration. The amount of liquid needed to disperse the tablets for oral suspension will depend on the dose (number of tablets prescribed) [see Dosage and Administration (2.7)].
Take OXBRYTA with or without food.
If a dose is missed or not consumed entirely, resume dosing the following day [see Dosage and Administration (2.7)].

PATIENT INFORMATION

| | |
|---|---|
| OXBRYTA ® | OXBRYTA ® |
| (ox brye ta) | (ox brye ta) |
| (voxelotor) | (voxelotor) |
| tablets | tablets for oral suspension |

```
What is OXBRYTA?
OXBRYTA is a prescription medicine used for the treatment of sickle cell disease in adults and
children 4 years of age and older.
It is not known if OXBRYTA is safe and effective in children with sickle cell disease below 4 years of
age.
Do not take OXBRYTA if you or your child have had an allergic reaction to voxelotor or
any of the ingredients in OXBRYTA. See the end of this leaflet for a list of the ingredients in
OXBRYTA.
Before taking OXBRYTA, tell your healthcare provider about all of your medical conditions,
including if you or your child:
  have liver problems
  are pregnant or plan to become pregnant. It is not known if OXBRYTA can harm your unborn
  baby.
  are breastfeeding or plan to breastfeed. It is not known if OXBRYTA can pass into your
  breastmilk and if it can harm your baby. Do not breastfeed during treatment with OXBRYTA and
  for at least 2 weeks after the last dose.
Tell your healthcare provider about all the medicines you or your child take, including
prescription and over-the-counter medicines, vitamins, and herbal supplements. Some medicines may
affect how OXBRYTA works. OXBRYTA may also affect how other medicines work and may affect
the results of certain blood tests.
Keep a list of all your medicines and show it to your healthcare provider.
How should I take OXBRYTA?
  Take OXBRYTA exactly as your healthcare provider tells you.
  Do not change your dose or stop taking OXBRYTA unless your healthcare provider tells you
  to. Your healthcare provider may change your dose if needed.
  Take your prescribed dose of OXBRYTA 1 time each day.
  Take OXBRYTA with or without food.
  OXBRYTA comes in two different dosage forms, OXBRYTA tablets and OXBRYTA
  tablets for oral suspension. Your healthcare provider will decide which dosage form you
  take based on your age, weight, and ability to swallow tablets.
    If you take OXBRYTA tablets: Swallow each OXBRYTA tablet whole. Do not cut, crush,
    or chew the tablets.
    If you take OXBRYTA tablets for oral suspension: See the detailed Instructions for Use
    on how to prepare and take your dose. You must mix the OXBRYTA tablets for oral
    suspension in room temperature clear drink, such as drinking water, clear soda, apple juice,
    clear electrolyte drinks, clear flavored drinks, or clear sports drinks, right before taking it. Do
    not swallow whole, cut, crush, or chew the tablets for oral suspension.
  Check to make sure you receive the correct dosage form of OXBRYTA each time your
  prescription isfilled to avoid taking the wrong medicine.
  Your healthcare provider may also prescribe a medicine called hydroxyurea during treatment with
  OXBRYTA.
  If you or your child miss a dose of OXBRYTA or if the entire dose is not taken, skip that
  dose and return to your normal dosing schedule the next day.
What should I avoid while taking OXBRYTA?
Do not take St. John's wort during treatment with OXBRYTA.
What are the possible side effects of OXBRYTA?
OXBRYTA can cause serious side effects, including:
  Serious allergic reactions. Tell your healthcare provider or get emergency medical help right
  away if you get:
```

| | |
|---|---|
| rash | shortness of breath (difficulty breathing) |
| hives | swelling of the face |

The most common side effects of OXBRYTA include:

| | |
|---|---|
| headache | nausea |
| diarrhea | rash or hives |
| stomach (abdominal) pain | fever |

The most common side effects of OXBRYTA in children ages 4 to less than 12 years of age
include:

| | |
|---|---|
| fever | stomach-area (abdominal) pain |
| vomiting | diarrhea |
| rash | headache |

```
These are not all the possible side effects of OXBRYTA.
Call your doctor for medical advice about side effects.
How should I store OXBRYTA?
  Store OXBRYTA between 68° F. to 86° F. (20° C. to 30° C.).
  The OXBRYTA bottle comes with a child-resistant closure.
  The OXBRYTA bottle may contain a desiccant canister to help keep your medicine dry (protect
  it from moisture) and a polyester coil. Do not eat the desiccant or polyester coil.
Keep OXBRYTA and all medicines out of the reach of children.
General information about the safe and effective use of OXBRYTA.
Medicines are sometimes prescribed for purposes other than those listed in a Patient Information leaflet.
Do not use OXBRYTA for a condition for which it was not prescribed. Do not give OXBRYTA to
other people, even if they have the same symptoms that you have. It may harm them. You can ask your
healthcare provider or pharmacist for information about OXBRYTA that is written for health
professionals.
What are the ingredients of OXBRYTA?
Active Ingredient: voxelotor
Inactive Ingredients:
OXBRYTA tablets: colloidal silicon dioxide, croscarmellose sodium, magnesium stearate,
microcrystalline cellulose, and sodium lauryl sulfate. The 500 mg tablet film coating contains:
polyethylene glycol 3350, polyvinyl alcohol, talc, titanium dioxide, and yellow iron oxide. The 300 mg
tablet film coating contains: black and red iron oxide, polyethylene glycol 3350, polyvinyl alcohol, talc,
and titanium dioxide.
OXBRYTA tablets for oral suspension: artificial grape flavor, colloidal silicon dioxide, croscarmellose
sodium, iron oxide pigment, magnesium stearate, microcrystalline cellulose, and sucralose.
Manufactured for: Global Blood Therapeutics, Inc. South San Francisco, CA 94080, USA.
OXBRYTA ®
© 2022 Global Blood Therapeutics, Inc. All rights reserved.
```

INSTRUCTIONS FOR USE
OXBRYTA ® [ox brye ta] (voxelotor)
tablets for oral suspension 300 mg

```
This Instructions for Use contains information on how to take OXBRYTA tablets for oral suspension.
Read this Instructions for Use before you or your child start taking OXBRYTA tablets for oral
suspension for the first time and each time you or your child get a refill. There may be new
information. This information does not take the place of talking to your healthcare provider about
your or your child's medical condition or treatment.
Talk to your healthcare provider or pharmacist if you have questions about how to take or give the
prescribed dose of OXBRYTA tablets for oral suspension.
Important Information You Need to Know Before Taking OXBRYTA Tablets for Oral
Suspension:
  Take OXBRYTA tablets for oral suspension exactly as your healthcare provider tells you.
  Your healthcare provider will tell you how many OXBRYTA tablets for oral suspension you will
  need for your or your child's dose.
  OXBRYTA comes in two different dosage forms. Each time you receive your or your child's
  prescription, check the bottle to make sure that you received the prescribed dosage form of
  OXBRYTA tablets for oral suspension. OXBRYTA tablets for oral suspension are light yellow
  to yellow, round shaped, and marked with “300 D” on one side. Contact your pharmacist or
  healthcare provider if you did not receive the correct dosage form.
  Do not swallow whole, cut, crush, or chew OXBRYTA tablets for oral suspension.
  You must first mix OXBRYTA tablets for oral suspension in room temperature clear drink, such
  as drinking water, clear soda, apple juice, clear electrolyte drinks, clear flavored drinks, or clear
  sports drinks, before swallowing.
  If you or your child miss a dose of OXBRYTA tablets for oral suspension or if the entire
  dose is not taken, skip that dose and return to the normal dosing schedule the next day.
  For more information about OXBRYTA tablets for oral suspension, see the Patient Information
  leaflet.
Gather supplies
You will need the following items to prepare the dose of OXBRYTA tablets for oral suspension
(not included with OXBRYTA tablets for oral suspension);
  a teaspoon
  a small cup
  room temperature clear drink, such as drinking water, clear soda, apple juice, clear electrolyte
  drinks, clear flavored drinks, or clear sports drinks.
You will also need:
  the prescribed number of OXBRYTA tablets for oral suspension needed for your dose.
Preparing a dose of OXBRYTA tablets for oral suspension
Step 1. Wash and dry your hands well before preparing the dose.
Step 2. Pour room temperature clear drink into the cup. The table below shows the amount of
      clear drink needed for your prescribed dose. You may add more clear drink if needed to
      mix the tablets.
```

| Number of OXBRYTA | |
|---|---|
| Tablets for Oral Suspension | Amount of Clear Drink |
| 1 | 1 teaspoon (5 mL) |
| 2 | 2 teaspoons (10 mL) |
| 3 | 3 teaspoons (15 mL) |
| 4 | 4 teaspoons (20 mL) |
| 5 | 5 teaspoons (25 mL) |
| 7 | 7 teaspoons (35 mL) |
| 8 | 8 teaspoons (40 mL) |

```
Step 3. Add the prescribed number of OXBRYTA tablets for oral suspension into the cup.
Step 4. Swirl the cup until the tablet(s) break apart (disperse) in the drink. Be careful not to spill
      the mixture.
      The tablet(s) will not completely dissolve. You will still see small tablet clumps in the
      mixture.
      If you spill any medicine, clean up the spill. Throw away the rest of the prepared medicine
      and make a new dose.
Step 5. Wait for 1 to 5 minutes.
Giving the dose
Step 6. Swirl the cup again. Take or give all of the prepared medicine right away.
      If giving to a child, make sure that your child is upright when drinking the medicine
      mixture.
Step 7. Add 1 or 2 teaspoons of room temperature clear drink to the cup to make sure the full dose
      is taken. Swirl the cup until the remaining medicine is mixed and take or give it right away.
      Repeat this step until no more medicine is left in the cup.
      After all the medicine is taken, you or your child may drink more water or any type of drink.
Step 8. Wash the teaspoon and cup with warm soap and water.
Storing OXBRYTA
      Store OXBRYTA between 68° F. to 86° F. (20° C. to 30° C.).
      The OXBRYTA bottle comes with a child-resistant closure.
      The OXBRYTA tablets for oral suspension bottle contains a polyester coil. Do not eat the
      polyester coil.
Keep OXBRYTA and all medicines out of the reach of children.
Disposing of OXBRYTA
When all the tablets in the bottle have been taken, throw away the bottle. Safely dispose of (throw
away) OXBRYTA that is out of date or no longer needed using your local household waste guidelines.
Manufactured for: Global Blood Therapeutics, Inc. South San Francisco, CA 94080, USA.
OXBRYTA ®
Global Blood Therapeutics, Inc. All rights reserved.
```

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A method of treating sickle cell disease in a patient in need thereof, comprising administering to the patient 600 mg to 1500 mg of voxelotor per day, wherein the patient is about 4 years old to less than about 12 years old, and the amount of voxelotor is determined by the patient's body weight.

2. The method of claim 1, wherein the patient's body weight is about 40 kg or greater, and the patient is administered 1500 mg of voxelotor per day.

3. The method of claim 1, wherein the patient's body weight is about 20 kg to less than about 40 kg, and the patient is administered 900 mg of voxelotor per day.

4. The method of claim 1, wherein the patient's body weight is about 10 kg to less than about 20 kg, and the patient is administered 600 mg of voxelotor per day.

5. A method of treating sickle cell disease in a patient in need thereof, comprising administering to the patient 300 mg to 1000 mg of voxelotor per day, wherein the patient has severe hepatic impairment, the patient is about 4 years old to less than about 12 years old, and the amount of voxelotor is determined by the patient's body weight.

6. The method of claim 5, wherein the patient's body weight is about 40 kg or greater, and the patient is administered 900 mg of voxelotor per day.

7. The method of claim 5, wherein the patient's body weight is about 40 kg or greater, and the patient is administered 1000 mg of voxelotor per day.

8. The method of claim 5, wherein the patient's body weight is about 20 kg to less than about 40 kg, and the patient is administered 600 mg of voxelotor per day.

9. The method of claim 5, wherein the patient's body weight is about 10 kg to less than about 20 kg, and the patient is administered 300 mg of voxelotor per day.

10. The method of any one of claims 1-9, wherein voxelotor is administered with food.

11. The method of any one of claims 1-9, wherein voxelotor is administered without food.

12. The method of any one of claims 5-11, wherein the patient has a Child-Pugh score of 10-15 points (Child-Pugh C).

13. The method of any one of claims 5-11, wherein the patient suffers from a chronic liver disease.

14. The method of any one of claims 5-11, wherein the patient suffers from a condition selected from the group consisting of liver fibrosis, cirrhosis, hepatocellular carcinoma, hepatic inflammation, hepatic steatosis, nonalcoholic fatty liver disease (NAFLD), nonalcoholic steatohepatitis (NASH), portal hypertension, hepatic encephalopathy, hepatitis, and any combination thereof.

15. The method of any one of claims 5-14, wherein voxelotor is administered to the patient once daily.

16. The method of any one of claims 5-15, wherein voxelotor is administered to the patient in an oral formulation.

17. The method of claim 16, wherein the oral formulation is tablet.

18. The method of any one of claims 1-17, wherein voxelotor is administered to the patient in a single oral dose of one tablet daily, two tablets daily, or three tablets daily.

19. A method of treating sickle cell disease in a patient in need thereof, comprising administering to the patient 600 mg to 1500 mg of voxelotor once daily, wherein the patient has mild or moderate hepatic impairment, the patient is about 4 years old to less than about 12 years old, and the amount of voxelotor is determined by the patient's body weight.

20. A method of concurrently administering voxelotor and a strong CYP3A4 inducer comprising administering to a patient a therapeutically effective amount of a strong CYP3A4 inducer and 2000 ng voxelotor orally once daily, wherein the patient is 12 years of age or older.

21. A method of concurrently administering voxelotor and a strong CYP3A4 inducer comprising administering to a patient a therapeutically effective amount of a strong CYP3A4 inducer and 900 mg to 2100 mg voxelotor orally once daily, wherein the patient is about 4 years old to less than about 12 years old, and the amount of voxelotor is determined by the patient's body weight.

22. The method of claim 21, wherein the patient's body weight is about 40 kg or greater, and the patient is administered 2000 mg of voxelotor per day.

23. The method of claim 21, wherein the patient's body weight is about 40 kg or greater, and the patient is administered 2100 mg of voxelotor per day.

24. The method of claim 21, wherein the patient's body weight is about 20 kg to less than about 40 kg, and the patient is administered 1200 mg of voxelotor per day.

25. The method of claim 21, wherein the patient's body weight is about 10 kg to less than about 20 kg, and the patient is administered 900 mg of voxelotor per day.

26. A method of administering voxelotor to a patient in need thereof, comprising administering to the patient a therapeutically effective amount of voxelotor, and advising the patient one or more of the following:
(a) advising the patient that a sensitive CYP3A4 substrate with a narrow therapeutic index, moderate CYP3A4 inducer, strong CYP3A4 inducer, fluconazole, or strong CYP3A4 inhibitor should be avoided or discontinued;
(b) advising the patient that co-administration of voxelotor with a sensitive CYP3A4 substrate with a narrow therapeutic index, moderate CYP3A4 inducer, strong CYP3A4 inducer, fluconazole, or strong CYP3A4 inhibitor can alter the therapeutic effect of voxelotor;
(c) advising the patient that co-administration of voxelotor and a moderate CYP3A4 inducer or strong CYP3A4 inducer can decrease voxelotor plasma concentrations and may lead to reduced efficacy;
(d) advising the patient that co-administration of voxelotor and a strong CYP3A4 inhibitor can increase voxelotor plasma concentrations and may lead to increased toxicity;
(e) advising the patient that co-administration of voxelotor with a sensitive CYP3A4 substrate with a narrow therapeutic index can increase the systemic exposure of the sensitive CYP3A4 substrate with a narrow therapeutic index;
(f) advising the patient to replace a strong CYP3A4 inhibitor or fluconazole with an alternative drug;
(g) if co-administration of a strong CYP3A4 inhibitor or fluconazole is unavoidable, decreasing the dose of voxelotor to 1000 mg per day;
(h) if co-administration of a moderate CYP3A4 inducer or strong CYP3A4 inducer is unavoidable, increasing the dose of voxelotor to 2500 mg per day;
(i) if co-administration of a CYP3A4 inducer is unavoidable and the patient is about 12 years old or older, increasing the dose of voxelotor to 2000 mg per day;
(j) if co-administration of a CYP3A4 inducer is unavoidable and the patient is about 4 years old to less than about 12 years old, increasing the dose of voxelotor to 900 mg to 2100 mg voxelotor mg per day depending on the patient's body weight;
(k) if co-administration of a strong CYP3A4 inducer is unavoidable and the patient is about 12 years old or older, increasing the dose of voxelotor to 2500 mg per day;
(l) if co-administration of a moderate CYP3A4 inducer is unavoidable and the patient is about 12 years old or older, increasing the dose of voxelotor to 2000 mg per day;
(m) if co-administration of a strong CYP3A4 inducer is unavoidable and the patient is about 4 years old to less than about 12 years old, increasing the dose of voxelotor to 900 mg to 2500 mg voxelotor mg per day depending on the patient's body weight;
(n) if co-administration of a moderate CYP3A4 inducer is unavoidable and the patient is about 4 years old to less than about 12 years old, increasing the dose of voxelotor to 900 mg to 2100 mg voxelotor mg per day depending on the patient's body weight; or
(o) if co-administration of a sensitive CYP3A4 substrate with a narrow therapeutic index is unavoidable, decreasing the dose of the sensitive CYP3A4 substrate with a narrow therapeutic index.

27. The method of any one of claims 1-19 and 21-26, wherein the patient may suffer from pyrexia, vomiting, rash, abdominal pain, diarrhea, and headache.

28. The method of any one of claims 1-19 and 21-26, wherein the patient may suffer from vomiting.

29. The method of any one of claims 1-19 and 21-28, wherein voxelotor is administered as an oral suspension.

30. The method of any one of the preceding claims, wherein a tablet of voxelotor is dispersed in a liquid before administration.

31. The method of claim 17, wherein one tablet of voxelotor is dispersed in 5 mL of liquid.

32. The method of claim 17, wherein two tablets of voxelotor are dispersed in 10 mL of liquid.

33. The method of claim 17, wherein three tablets of voxelotor are dispersed in 15 mL of liquid.

34. The method of claim 17, wherein four tablets of voxelotor are dispersed in 20 mL of liquid.

35. The method of claim 17, wherein four tablets of voxelotor are dispersed in 20 mL of liquid.

36. The method of claim 17, wherein five tablets of voxelotor are dispersed in 25 mL of liquid.

37. The method of claim 17, wherein seven tablets of voxelotor are dispersed in 35 mL of liquid.

38. The method of claim 17, wherein eight tablets of voxelotor are dispersed in 40 mL of liquid.

39. A method of concurrently administering voxelotor and a moderate CYP3A4 inducer comprising administering to a patient a therapeutically effective amount of a moderate CYP3A4 inducer and 2000 mg voxelotor orally once daily, wherein the patient is 12 years of age or older.

40. A method of concurrently administering voxelotor and a strong CYP3A4 inducer comprising administering to a patient a therapeutically effective amount of a strong CYP3A4 inducer and 900 mg to 2500 mg voxelotor orally once daily, wherein the patient is about 4 years old to less than about 12 years old, and the amount of voxelotor is determined by the patient's body weight.

41. The method of claim 40, wherein the patient's body weight is about 40 kg or greater, and the patient is administered 2500 mg of voxelotor per day.

42. The method of claim 40, wherein the patient's body weight is about 40 kg or greater, and the patient is administered 2400 mg of voxelotor per day.

43. The method of claim 40, wherein the patient's body weight is about 20 kg to less than about 40 kg, and the patient is administered 1500 mg of voxelotor per day.

44. The method of claim 40, wherein the patient's body weight is about 10 kg to less than about 20 kg, and the patient is administered 900 mg of voxelotor per day.

45. A method of concurrently administering voxelotor and a moderate CYP3A4 inducer comprising administering to a patient a therapeutically effective amount of a moderate CYP3A4 inducer and 900 mg to 2100 mg voxelotor orally once daily, wherein the patient is about 4 years old to less than about 12 years old, and the amount of voxelotor is determined by the patient's body weight.

46. The method of claim 45, wherein the patient's body weight is about 40 kg or greater, and the patient is administered 2000 mg of voxelotor per day.

47. The method of claim 45, wherein the patient's body weight is about 40 kg or greater, and the patient is administered 2100 mg of voxelotor per day.

48. The method of claim 45, wherein the patient's body weight is about 20 kg to less than about 40 kg, and the patient is administered 1200 mg of voxelotor per day.

49. The method of claim 45, wherein the patient's body weight is about 10 kg to less than about 20 kg, and the patient is administered 900 mg of voxelotor per day.

50. The method of any one of the preceding claims, further comprising administering to the patient hydroxyurea.
